(19) United States
(12) Patent Application Publication
(10) Pub. No.: US 2005/0249742 A1
(43) Pub. Date: Nov. 10, 2005

(54) Compositions and methods for modulating a cytotoxic T lymphocyte immune response

(75) Inventors: Ruth M. Ruprecht, Brookline, MA (US); Shisong Jiang, Chestnut Hill, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Dana-Farber Cancer Institute, Inc., Boston, MA (US)

(21) Appl. No.: 11/022,562

(22) Filed: Dec. 22, 2004

Related U.S. Application Data
(63) Continuation of application No. PCT/US03/20322, filed on Jun. 27, 2003.
(60) Provisional application No. 60/392,718, filed on Jun. 27, 2002.

(51) Int. Cl. A61K 39/385; A61K 39/00

(52) U.S. Cl. 424/185.1

(57) ABSTRACT

The present invention provides compositions and methods for the treatment and prevention of immune disorders.

### RELATED APPLICATIONS

This application is a continuation of PCT/US03/20322, filed Jun. 27, 2003 which claims the benefit of U.S. Provisional Application Ser. No. 60/392,718, filed Jun. 27, 2002, the entire contents of which are incorporated herein by this reference.

### BACKGROUND OF THE INVENTION

The initiation of an immune response against a specific antigen in mammals is brought about by the presentation of that antigen to T lymphocytes. An antigen is presented to T lymphocytes in the context of a major histocompatablity (MHC) complex (also referred to as HLA in humans and H-2 in mice). The three-dimensional structure of the MHC includes a groove or cleft into which the presented antigen fits. When an appropriate receptor on a T lymphocyte interacts with the MHC/antigen complex on an APC in the presence of necessary co-stimulatory signals, the T lymphocyte is stimulated, triggering various aspects of the well characterized cascade of immune system activation events, including induction of cytotoxic T lymphocyte (CTL) function, induction of B lymphocyte function and stimulation of cytokine production (see, e.g. Roitt, I and Delves, P. Roitt's Essential Immunology, 10^{th} Ed., Boston, Blackwell Science, 2002; Abbas, A. et al. Cellular and Molecular Immunology, W.B. Saunders Company, Philadelphia, 1991; Silverstein, A. A History of Immunology, San Diego, Academic Press, 1989).

There are two basic classes of MHC molecules in mammals, MHC Class I and MHC Class II. Both classes are large complexes formed by association of two separate proteins. MHC Class I molecules present antigen to CD8-positive T lymphocytes, which then become activated and can kill the antigen presenting cell directly. Class I MHC molecules generally receive peptides from endogenously synthesized proteins, such as an infectious virus, in the endoplasmic reticulum at around the time of their synthesis (see, e.g., Williams, A. et al. (2002) Tissue Antigens 59:3; Konig, R. (2002) Curr. Opin. Immunol. 14:75; Anfossi, N. et al. (2001) Immunol. Rev. 14:75; Gao G. and Jakobsen B (2000) Immunol. Today 21:630; Watts, C and Powis, S. (1999) Rev. Immunogenet. 1:60 and Natarajan, K. et al. (1999) Rev. Immunogenet. 1:32).

MHC Class II molecules present antigen to CD4-positive T helper lymphocytes (Th cells). Once activated, Th cells contribute to the activation of CTLs and B lymphocytes via physical contact and cytokine release. Unlike MHC Class I molecules, MHC class II molecules bind exogenous antigens which have been internalized via non-specific or specific endocytosis. Around the time of synthesis, MHC Class II molecules are blocked from binding endogenous antigen, and instead bind the invariant chain protein (Ii). These MHC Class II-Ii protein complexes are transported from the endoplasmic reticulum to a post-Golgi compartment where Ii is released by proteolysis and exogenous antigenic peptides are bound (see, e.g., Villadangos, J. (2001) Mol. Immunol. 38:329; Alfonso, C. and Karlsson, L. (2000) Ann. Rev. Immunol. 18:113; Viret, C. and Janeway Jr., C. (1999) Rev. Immunogenet. 1:91; Diabata et al. (1994) Molecular Immunology 31:255 and Xu et al. (1994) Molecular Immunology 31:723).

MHC Class I and MHC Class II molecules have a distinct distribution among cells. Almost all nucleated cells express MHC Class I molecules, although the level of expression varies between cell types. Cells of the immune system express abundant MHC Class I on their surfaces, while liver cells express relatively low levels. Non-nucleated cells express little or no MHC Class I. MHC Class II molecules are highly expressed on B lymphocytes, dendritic cells and macrophages, but not on other tissue cells. However, many other cell types can be induced to express MHC Class II molecules by exposure to cytokines (see, e.g. Roitt, I and Delves, P. Roitt's Essential Immunology, 10^{th} Ed., Boston, Blackwell Science, 2002; Abbas, A. et al. Cellular and Molecular Immunology, W.B. Saunders Company, Philadelphia, 1991; Silverstein, A. A History of Immunology, San Diego, Academic Press, 1989).

Cytotoxic T lymphocytes (CTLs) are restricted in their activity by recognizing a specific histocompatability complex (MHC) antigen on the surface of the target cell, as well as a peptide bound in a cleft of the MHC antigen. The foreign antigen may be present as a result of transplantation from an allogeneic host, viral or bacterial infection, mutation, neoplasia, or the like. The involvement of the MHC protein appears to be essential to the attack by CTLs against the cell which includes the foreign antigen. By monitoring the presence of foreign antigens, the CTLs are able to destroy cells, which if otherwise allowed to proliferate, might result in the proliferation of pathogens or neoplastic cells (see, e.g. Roitt, I and Delves, P. Roitt's Essential Immunology, 10^{th} Ed., Boston, Blackwell Science, 2002; Rhodes, D. and Trowsdale, J. (1999) Rev. Immunogenet. 1:21; and Yu, C. (1998) Exp. Clin. Immunogenet. 15:213).

The unique capability of CTLs to kill infected and/or cancerous cells has led researchers to try and develop strategies for using CTLs in the designing of vaccines for the treatment of diseases, i.e. pathogenic infections and cancer. However, vaccines of killed pathogens or soluble proteins are not effective in the induction of the CTL response. Moreover, naked DNA, live vectors and attenuated viruses, which are effective CTL inducers, are genetic material and potentially pose a serious health hazard, especially in the case of viruses such as human immunodeficiency virus (HIV) and Ebola virus (see, e.g., Baba, T. et al. (1999) Nat. Med. 5:194).

This problem was thought to be solved with the finding that specific T-cell epitopes could be synthetically designed and produced. Townsend et al. demonstrated that epitopes of influenza nucleoprotein could be defined by short synthetic peptides and thus included in potential vaccine candidates (Townsend, A. et al. (1986) Nature 324:575). However, success using synthetic peptides has been limited. Documented cases that describe the use of synthetic peptides, relating to influenza, Sendai and lymphocyte choriomeningitis viruses, for use in the in vivo priming of CTLs have presented many problems (see, e.g., Kast, W. et al., (1991) Immunol. Lett. 30:229; Aichele, P. et al., (1990) J. Exp. Med. 171:1815; Deres, K. et al. (1989) Nature 342:561). In each of the above cases, the immunization protocols proved to be cumbersome, requiring either modifications of peptides or multiple immunizations to demonstrate CTL activity, and difficulty in rapidly screening large numbers of candidate substances.

Moreover, the use of single epitopic peptides has been shown to only generate CTL responses in a small group of individuals, i.e. those individuals who have matched MHC antigens, thus decreasing the effectiveness and usefulness of the vaccine. Although the use of multiple epitopic peptides has been shown to increase the size of the population who will benefit from the vaccine (Hanke, T and McMichael, A. (2000) Nat. Med. 6:951), it remains difficult and labor intensive to accurately predict from a sequence of an antigenic protein how the protein will be processed and which peptide portions will bind HLA class I molecules and be presented to CTLs.

The present invention provides an effective method of modulating, e.g., inducing, an immune response, e.g., a CTL-mediated immune response, which avoids may of the problems associated with the previously suggested methods. Specifically, the present invention allows for the development of vaccines that are capable of inducing antigen-specific immune responses in subjects of varying genetic backgrounds without the labor intensive task of determining immunostimulatory epitopes.

### SUMMARY OF THE INVENTION

The present invention provides, at least in part, methods and compositions for the treatment of immune disorders, such as, for example, viral, bacterial and parasitic infections, prion diseases, neoplastic diseases and protection against toxins. The invention is based on the discovery that overlapping synthetic peptide formulations (OSPFs) of the present invention are able to modulate, e.g., induce, immune responses, such as cytotoxic T lymphocyte (CTL)-mediated response and antibody-associated immune responses, thus indicating a wide applicability for human and veterinary applications.

Accordingly, the present invention provides a method of modulating, e.g. inducing, an immune response by administering to a subject, e.g., a vertebrate, such as a human, an effective amount of an OSPF. The OSPF of the present invention includes a combination of single chain peptides that correspond to an amino acid sequence of a protein of interest, such that the single chain peptide is a length represented by Y, wherein Y is at least 7 to (X-1), and X represents the number of amino acids of the protein of interest, where at least 1 single chain peptide overlaps with another single chain peptide by a length represented by Z, wherein Z is 1 to (Y-1), such that the length of the single chain peptide is able to be internalized by, e.g., phagocytosis, receptor-mediated endocytosis, and the like, by a MHC bearing cell, i.e., a MHC class I- or MHC class II-bearing cell, and be presented by an MHC molecule to a T cell.

In another embodiment, the OSPFs of the present invention are not overlapping, but instead are adjoining. Therefore, in this embodiment, the OSPF of the present invention includes a combination of single chain peptides that correspond to an amino acid sequence of a protein of interest, such that the single chain peptide is a length represented by Y, wherein Y is at least 7 to (X-1) and X represents the number of amino acids of the protein of interest, such that the length of the single chain peptide is able to be internalized, e.g., phagocytosis, receptor-mediated endocytosis, and the like, by a MHC-bearing cell, i.e. a MHC Class I- or MHC Class II-bearing cell, and be presented by an MHC molecule to a T cell.

In one embodiment, the immune response is a Th1-mediated immune response, such as a CTL-mediated immune response. In another embodiment, the immune response is a Th2-mediated immune response, such as an antibody-associated immune response.

In one embodiment, Y is at least 7, 8, 9, 10, 11, 12, 13, 14, 15, 16, 17, 18, 19, 20, 21, 22,23, 24, 25, 26, 27, 28, 29, or 30 amino acids.

In another embodiment, Z is at least 1, 2, 3, 4, 5, 6, 7, 8, 9, 10, 11, 12, 13, 14, 15, 16, 17, 18, 19, 20,21, 22, 23, 24, 25, 26, 27, 28, or 29 amino acids.

In other aspects, the invention pertains to a method of treating or preventing an OSPF-associated disorder in a subject. The method includes administering to the subject an effective amount of an OSPF of the present invention, thereby treating or preventing the OSPF-associated disorder in the subject. By “OSPF-associated disorder” is meant any disease, disorder or condition which can be treated or prevented through the modulation of an immune response. Examples of OSPF-associated disorders include, but is not limited to, viral infections due to viruses (e.g., Ebola virus, hepatitis C, HIV, e.g., HIV-1 and HIV-2, RSV, monkeypox, and SARS coronavirus, bacterial infections due to bacteria (e.g., anthrax, Listeria monocytogenes, Legionella and mycobacterium such as tuberculosis), parasitic infections (e.g. malaria), protection against toxins (e.g., shigella toxin, toxin botulinum and tetanus toxin), parasitic infections due to parasites (e.g., Plasmodium, Trypanosoma, Schistosoma and Toxoplasmosis), prions and neoplastic diseases (e.g., breast, colon, non-small cell lung, head and neck, colorectal, lung, prostate, ovary, renal, melanoma, gastrointestinal (e.g., pancreatic and stomach) cancer and osteogenic sarcoma).

In yet another embodiment, the protein of interest can be any protein associated with an OSPF-associated disorder, including, but not limited to, HIV Gag protein (SEQ ID NO:339); SIV Envelope protein (SEQ ID NO:340); anthrax toxins translocating protein (protective antigen precursor [PA]) (SEQ ID NO:209); Ebola virus nucleoprotein (SEQ ID NO:210); hepatitis C virus (HCV) polyprotein (SEQ ID NO:21 1); melanoma antigen p15 (SEQ ID NO:212); human Her2/neu protein (SEQ ID NO:213); respiratory syncytial virus (RSV) fusion protein (SEQ ID NO:214); HIV-2 gp41 protein (SEQ ID NO:215); HIV-2 GAG protein (SEQ ID NO:216); HIV-2 envelope (env) protein (SEQ ID NO:217); HIV-1 vpu protein (SEQ ID NO:218); HIV-1 envelope (env) protein (SEQ ID NO: 219); HIV-1 Tat interactive protein 2 (SEQ ID NO:220); HIV-1 reverse transcriptase (SEQ ID NO:221) and HIV-1 nef protein (SEQ ID NO:222); circumsporozoite protein precursor (SEQ ID NO:223); circumsporozoite protein II (SEQ ID NO:224); pertussis-like toxin subunit (SEQ ID NO:225); S. aureus enterotoxin A (SEQ ID NO:226); E. coli enterotoxin A (SEQ ID NO:227); C. difficile enterotoxin A (SEQ ID NO:228); B. cereus enterotoxin A (SEQ ID NO:229); pertussis toxin subunit 3 (SEQ ID NO:230) ); SARS coronavirus (Frankfurt 1) envelope protein E (SEQ ID No:231); Human metapneumovirus fusion protein (SEQ ID NO:232); SARS coronavirus matrix protein (SEQ ID NO: 233); coronavirus nucleocapsid protein (SEQ ID NO: 234); and SARS coronavirus (Frankfurt 1) spike protein S (SEQ ID NO: 235). For example, OSPFs for HIV-1 Gag include the peptides set forth as SEQ ID NO:1-122 and/or SEQ ID NO:236-335 and OSPFs for SIV Envelope protein include the peptides set forth as SEQ ID NO: 123-206 and/or 336-338.

In another aspect, the invention provides a vaccine for immunizing a subject against an OSPF-associated disorder, wherein the vaccine comprises an OSPF of the present invention and a pharmaceutically-acceptable carrier. In another aspect, the invention provides a pharmaceutical composition comprising an OSPF of the present invention and a pharmaceutically acceptable carrier. In yet another aspect, the invention features a kit for immunizing a subject against an OSPF-associated disorder, wherein the kit comprises an OSPF of the present invention and may further comprise instructions for use.

In yet another aspect, the invention features a vaccine adjuvant which comprises an OSPF of the present invention and a pharmaceutically acceptable carrier which may be used to enhance the efficacy of a vaccine.

### BRIEF DESCRIPTION OF THE DRAWINGS

FIG. 1a-1c are graphs depicting the CTL activity induced by OSPF-HIV Gag in BALB/c and C57BL/6 mice.

FIGS. 2a and 2b are graphs depicting T cell proliferation induced by OSPF-HIV Gag in BALB/c and C57BL/6 mice

FIGS. 3a and 3b are graphs depicting the CTL activity induced by OSPF-SIV ex vivo by human dendritic cells and autologous PBMCs, as assessed by ELISPOT™ and ⁵¹Cr release assays, respectively.

### DETAILED DESCRIPTION OF THE INVENTION

Definitions

Before further description of the present invention, and in order that the invention may be more readily understood, certain terms are first defined and collected here for convenience.

The term “overlapping synthetic peptide formulation” or “OSPF” refers to a combination of single chain peptides which correspond to an amino acid sequence of a protein of interest, represented by Y, wherein Y is at least 7 to (X-1) and X represents the number of amino acids of the protein interest where at least 1 single chain peptide overlaps with another single chain peptide by a length represented by Z, wherein Z is 1 to (Y-1). The length of the single chain peptide must be such that internalization, e.g., phagocytosis, receptor-mediated endocytosis, and the like, of the single chain peptide by a MHC-bearing cell, i.e. a MHC-Class I- or MHC Class II-bearing cell, can occur. Preferably, the cell is a MHC Class I-bearing cell. Furthermore, the OSPF must be of a length to allow presentation by a MHC molecule to a T cell. In certain embodiments, Y is at least 7, 8, 9, 10, 11, 12, 13, 14, 15, 16, 17, 18, 19, 20, 21, 22, 23, 24, 25, 26, 27, 28, 29 or 30 amino acids in length. In other embodiments, the length of Z is at least 1, 2, 3, 4, 5, 6, 7, 8, 9, 10, 11, 12, 13, 14, 15, 16, 17, 18, 19, 20, 21, 22, 23, 24,25, 26, 27, 28 or 29 amino acids.

In another embodiment of the invention, the OSPF refers to a combination of single chain peptides that correspond to a protein of interest and are represented by Y, wherein Y is 1 to (X-1), where X represents the number of amino acids of the protein of interest. The length of the single chain peptide must be such that internalization, e.g., phagocytosis, receptor-mediated endocytosis, and the like, of the single chain peptide by a MHC-bearing cell, i.e. a MHC Class I- or MHC Class II-bearing cell, can occur. Furthermore, the OSPF must be of a length to allow presentation by a MHC molecule to a T cell. Preferably, the cell is a MHC Class I-bearing cell. In certain embodiments, Y is at least 7, 8, 9, 10, 11, 12, 13, 14, 15, 16, 17, 18, 19, 20, 21, 22, 23, 24, 25, 26, 27, 28, 29 or 30 amino acids in length.

As used herein, the term “combination” or “a combination of” refers to two or more single chain peptides.

The term “peptide” or “single chain peptide” or “polypeptide” is used in its broadest sense, i.e., any polymer of amino acids (dipeptide or greater) linked through peptide bonds. Thus, the term “peptide” includes proteins, oligopeptides, protein fragments, mutants, fusion proteins and the like. The term “protein” is used herein to designate a naturally occurring polypeptide. Peptides of the present invention can be made synthetically, using techniques that are known in the art, or encoded by a nucleic acid, such as DNA or RNA.

The present invention also includes a recombinant molecule comprising a nucleic acid sequence encoding an OSPF(s), operatively linked to a vector capable of being expressed in a host cell. As used herein, “operatively linked” refers to insertion of a nucleic acid sequence into an expression vector in such a manner that the sequence is capable of being expressed when transformed into a host cell. As used herein, an “expression vector” is an RNA or DNA vector capable of transforming a host cell and effecting expression of an appropriate nucleic acid sequence, preferably replicating within the host cell. An expression vector can be either prokaryotic or eukaryotic, and typically is a virus or a plasmid. Suitable host cells can be any cells that are capable of producing the peptides of the present invention. Such host cells include, but are not limited to, bacterial, fungal, insect and mammalian cells. Host cells of the present invention can also be cells which naturally express an MHC molecule, or are capable of expressing an MHC molecule, and can produce the peptides of the present invention and present them on a MHC molecule. Suitable host cells also include mammalian cells which express MHC molecules on their cell surface and are capable of stimulating an immune response. Examples include, but are not limited to, T cells and antigen presenting cells, such as B cells, dendritic cells, and macrophages. Other examples include non-immune cells which express MHC class I molecules on the cell surface, and include, but are not limited to, fibroblasts, epithelial cells and endothelial cells.

The term “overlapping synthetic peptide formulation (OSPF)-associated disorder” includes any disease, disorder or condition which can be treated or prevented through the modulation, e.g., up-regulation or down-regulation, of an immune response. In certain embodiments, the immune response is a Th-1-mediated immune response, such as a CTL-mediated immune response. In another embodiment, the immune response is a Th2-mediated immune response, such as an antibody-associated immune response. In certain embodiments, OSPF-associated disorders include disorders in which CTL activity is low, aberrant or absent. In other embodiments, the OSPF-associated disorder is an intracellular infection, e.g., a viral infection, a bacterial infection, a parasitic infection, toxic poisoning, prion disease and a neoplastic disease.

The term “protein of interest” refers to any protein associated with an OSPF-associated disorder. Examples of proteins of interest include, but are not limited to, HIV Gag protein (SEQ ID NO:239) SIV Envelope protein (SEQ ID NO:240); anthrax toxins translocating protein (protective antigen precursor [PA]) (SEQ ID NO:209); Ebola virus nucleoprotein (SEQ ID NO:210); hepatitis C virus (HCV) polyprotein (SEQ ID NO:211); melanoma antigen p15 (SEQ ID NO:212); human Her2/neu protein (SEQ ID NO:213); respiratory syncytial virus (RSV) fusion protein (SEQ ID NO:214); HIV-2 gp41 protein (SEQ ID NO:215); HIV-2 GAG protein (SEQ ID NO:216); HIV-2 envelope (env) protein (SEQ ID NO:217); HIV-1 vpu protein (SEQ ID NO:218); HIV-1 envelope (env) protein (SEQ ID NO: 219); HIV-1 Tat interactive protein 2 (SEQ ID NO:220); HIV-1 reverse transcriptase (SEQ ID NO:221) and HIV-1 nef protein (SEQ ID NO:222); circumsporozoite protein precursor (SEQ ID NO:223); circumsporozoite protein II (SEQ ID NO:224); pertussis-like toxin subunit (SEQ ID NO:225); S. aureus enterotoxin A (SEQ ID NO:226); E. coli enterotoxin A (SEQ ID NO:227); C. difficile enterotoxin A (SEQ ID NO:228); B. cereus enterotoxin A (SEQ ID NO:229); pertussis toxin subunit 3 (SEQ ID NO:230) ); SARS coronavirus (Frankfurt 1) envelope protein E (SEQ ID No:231); Human metapneumovirus fusion protein (SEQ ID NO:232); SARS coronavirus matrix protein (SEQ ID NO: 233); coronavirus nucleocapsid protein (SEQ ID NO: 234); and SARS coronavirus (Frankfurt 1) spike protein S (SEQ ID NO: 235). For example, OSPFs for HIV-1 Gag include the peptides set forth as SEQ ID NO:1-122 and/or SEQ ID NO:236-335 and OSPFs for SIV Envelope protein include the peptides set forth as SEQ ID NO:123-206 and/or 336-338.

The methods of the present invention are effective for preventing, treating or eliminating disease caused by a variety of viruses such as, but not limited to, HIV, e.g., HIV-1 and HIV-2, human herpes viruses, cytomegalovirus (esp. Human), Rotavirus, Epstein-Barr virus, Varicella Zoster Virus, hepatitis viruses, such as hepatitis B virus, hepatitis A virus, hepatitis C virus and hepatitis E virus, coronaviruses (e.g SARS coronavirus), orthopoxviruses (e.g monkeypox and smallpox), paramyxoviruses: Respiratory Syncytial virus, parainfluenza virus, measles virus, mumps virus, human papilloma viruses (for example HPV6, 11, 16, 18 and the like), flaviviruses (e.g. Yellow Fever Virus, Dengue Virus, Tick-borne encephalitis virus, Japanese Encephalitis Virus) or influenza virus.

The methods of the present invention are effective for preventing, treating or eliminating disease caused by a variety of bacterial organisms, including gram-positive and gram-negative bacteria. Examples include, but are not limited to, Neisseria spp, including N. gonorrhea and N. meningitidis, Streptococcus spp, including S. pneumoniae, S. pyogenes, S. agalactiae, S. mutans; Haemophilus spp, including H. influenzae type B, non typeable H. influenzae, H. ducreyi; Moraxella spp, including M catarrhalis, also known as Branhamella catarrhalis; Bordetella spp, including B. pertussis, B. parapertussis and B. bronchiseptica; Mycobacterium spp., including M. tuberculosis, M. bovis, M. leprae, M. avium, M. paratuberculosis, M. smegmatis; Legionella spp, including L. pneumophila; Escherichia spp, including enterotoxic E. coli, enterohemorragic E. coli, enteropathogenic E. coli; Vibrio spp, including V. cholera, Shigella spp, including S. sonnei, S. dysenteriae, S. flexnerii; Yersinia spp, including Y. enterocolitica, Y. pestis, Y. pseudotuberculosis, Campylobacter spp, including C. jejuni and C. coli; Salmonella spp, including S. typhi, S. paratyphi, S. choleraesuis, S. enteritidis; Listeria spp., including L. monocytogenes; Helicobacter spp, including H. pylori; Pseudomonas spp, including P. aeruginosa, Staphylococcus spp., including S. aureus, S. epidermidis; Enterococcus spp., including E. faecalis, E. faecium; Clostridium spp., including C. tetani, C. botulinum, C. difficile; Bacillus spp., including B. anthracis; Corynebacterium spp., including C. diphtheriae; Borrelia spp., including B. burgdorferi, B. garinii, B. afzelii, B. andersonii, B. hermsii; Ehrlichia spp., including E. equi and the agent of the Human Granulocytic Ehrlichiosis; Rickettsia spp, including R. rickettsii; Chlamydia spp., including C. trachomatis, C. neumoniae, C. psittaci; Leptira spp., including L. interrogans; Treponema spp., including T. pallidum, T. denticola, T. hyodysenteriae. Preferred bacteria include, but are not limited to, Listeria, mycobacteria, mycobacteria (e.g., tuberculosis), Anthrax, Salmonella and Listeria monocytogenes.

The methods of the present invention are effective for preventing, treating or eliminating disease caused by a variety of protozoal and parasitic organisms such as, but not limited to, Anaplasma, Babesia, Balantidium, Besnoitia, Chlamydia, Coccidia, Cryptosporondium, Cytauxzoon, Eimeria Entamoeba, Eperythrozoon, Erlichia, Giardia, Haemobartonella, Hammondia, Isopora, Leishmania, Neorickettsia, Plasmodium, Pneumocystis, Rickettsia, Schistosoma, Sarcocystis, Theileria, Thrichinella, Toxoplasma, Trichomonas, Trypanosoma, Unicaria, Dipylidium, Echinococcuse, Taenia, Ancylostoma, Ascaris, Enterobius, Strongyloides, Strongylus, Toxocara, Toxascaris and Trichuris. The methods are particularly useful for treating blood-borne protozoal and parasitic diseases.

As used herein, the term “state of toxicity” or “toxin-induced condition” refers to the quality of being poisonous, i.e. that caused by a poison or toxin. As used in the art, this term also refers to the degree of virulence of a toxic microbe or of a poison. By “toxin” it is meant a poisonous substance of biological origin, which necessarily excludes synthetic toxins which are not encoded by a living organism. The toxins are usually, but are not necessarily, proteins. The methods of the present invention for treating and preventing a toxin-related OSPF disorder are effective for preventing, treating or eliminating toxicity caused by a variety of toxins. Nonlimiting examples of protein toxins include botulin, perfringens toxin, pertussis, mycotoxins, shigatoxins, staphylococcal enterotoxin B, tetanus, ricin, cholera, aflatoxins, diphtheria, T2, seguitoxin, saxitoxin, abrin, cyanoginosin, alphatoxin, tetrodotoxin, aconotoxin, snake venom, scorpion venom and other spider venoms. A nonlimiting example of a non-protein toxin is tricothecene (T-2). Toxin-producing microorganisms of interest include, but are not limited to: Corynebacterium diphtheriae, Staphylococci, Salmonella typhimuium, Shigellae, Pseudomonas aeruginosa, Vibrio cholerae, Clostridium botulinum, and Clostridium tetani. A nonlimiting example of a toxin producing plant is Ricinus communis, and of a fungus producing a toxin is Aspergillus favus.

The methods of the present invention are effective for preventing, treating or eliminating disease caused by prions, such as, but not limited to, familial Creutzfeldt-Jakob disease, Gerstmann-Straussler-Scheinker disease, bovine spongiform encephalopathy (BSE), scrapie and fatal familial Insomnia. As used herein, the term “prion” or “prion disease” refers to a group of transmissible spongiform encephalopathies or TSE. TSEs are caused by abnormalities of the prion protein (PrP). For example, Creutzfeldt-Jakob disease is caused by the conversion of the normal protease-sensitive PrP isoform, designated PrP(C), to a protease resistant isoform, designated PrP(Sc). The change of PrPC into PrPSc can occur spontaneously, however, it can also be induced by PrPSc. PrP(Sc) forms into an infectious particle, named a ‘prion’ that can transmit the disease. The process by which prions proceed to the central nervous system (CNS) following peripheral uptake is referred to as neuroinvasion Accumulation of PrP(Sc) in the brain causes degenerative disorders affecting the CNS leading to neurodegeneration.

As used herein, the term “neoplastic disease” is characterized by malignant tumor growth or in disease states characterized by benign hyperproliferative and hyperplastic cells. The common medical meaning of the term “neoplasia” refers to “new cell growth” that results as a loss of responsiveness to normal growth controls, e.g., neoplastic cell growth.

As used herein, the terms “hyperproliferative”, “hyperplastic”, malignant” and “neoplastic” are used interchangeably, and refer to those cells in an abnormal state or condition characterized by rapid proliferation or neoplasia. The terms are meant to include all types of hyperproliferative growth, hyperplastic growth, cancerous growths or oncogenic processes, metastatic tissues or malignantly transformed cells, tissues, or organs, irrespective of histopathologic type or stage of invasiveness. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be either benign, premalignant or malignant.

The terms “neoplasia,” “hyperplasia,” and “tumor” are often commonly referred to as “cancer,” which is a general name for more than 100 diseases that are characterized by uncontrolled, abnormal growth of cells. Examples of cancer include, but are not limited to: breast; colon; non-small cell lung, head and neck; colorectal; lung; prostate; ovary; renal; melanoma; and gastrointestinal (e.g., pancreatic and stomach) cancer; and osteogenic sarcoma.

The term “tumor antigen” as used herein relates to any antigen expressed on a tumor cell, including but not limited to, Mucinl, carcinoembryonic antigen, oncofetal antigens and tumor-associated antigens. Also included in this definition are any antigens expressed by tumor cells that are encoded by a single DNA strand.

The terms “induce”, “inhibit”, “potentiate”, elevate”, “increase” “decrease” or the like, denote quantitative differences between two states, refer to at least statistically significant differences between the two states. For example, “an amount effective to inhibit growth of hyperproliferative cells” means that the rate of growth of the cells will at least statistically significantly different from the untreated cells. Such terms are applied herein to, for example rates of cell proliferation.

As used herein, the term “subject” is intended to include all vertebrates, i.e. human and non-human animals. The term “non-human animals” of the invention includes, but is not limited to, mammals, rodents, mice, and non-mammals, such as non-human primates, sheep, dog, horse, cow, chickens, amphibians, reptiles and the like. In one embodiment, the subject is a mammal, e.g., a primate, e.g., a human. In another embodiment, human animals include a human patient suffering from or prone to suffering from an OSPF-associated disorder.

The term “treatment” or “treating” as used herein refers to either (1) the prevention of a disease or disorder (prophylaxis), or (2) the reduction or elimination of symptoms of the disease or disorder (therapy).

The terms “prevention”, “prevent” or “preventing” as used herein refers to inhibiting, averting or obviating the onset or progression of a disease or disorder (prophylaxis).

As used herein, the terms “immune” and “immunity” refers to the quality or condition of being able to resist a particular disease.

The terms “immunize” and “immunization,” as used herein, refer to the act of making a subject (1) not susceptible to a disease or disorder; or (2) less responsive to a disease or disorder; or (3) have an increased degree of resistance to a disease or disorder.

The term “MHC-bearing cell” refers to any cell which expresses an MHC molecule, i.e. MHC Class I or Class II molecule, on the cell surface. In humans, almost all nucleated cells express MHC Class I molecules, although the level of expression varies between cell types. Cells of the immune system express abundant MHC Class I on their surfaces, while liver cells express relatively low levels. MHC Class II molecules are primarily expressed on immune cells, particularly antigen presenting cells, i.e., B cells, dendritic cells, monocytes and macrophages. However, many other cell types can be induced to express MHC Class II molecules and are also meant to be within the scope of the invention. MHC molecules often have different names between vertebrates. For example, MHC is often referred to as HLA in humans and H-2 in mice. These differences in nomenclature are intended to be within the scope of the present invention.

The term “immune cell” includes cells of the immune system which are capable of expressing, producing or secreting cytokines that regulate an immune response, for example a type-1 (Th1) or type-2 (Th2) immune response. Preferred immune cells include human immune cells. Exemplary preferred immune cells include, but are not limited to, macrophages, dendritic cells, T cells, B cells and neutrophils.

As used herein, the term “T cell” (i.e. T lymphocytes) is intended to include all cells within the T cell lineage, including thymocytes, immature T cells, mature T cells (including T cells bearing the surface markers CD4 and/or CD8) and the like, from a mammal (e.g. human or mouse). Preferably, the T cell is a CD8⁺ T cell, also referred to herein as a “cytotoxic T lymphocyte” or “CTL”, or a CD4⁺ T cell, also referred to herein as a “helper T lymphocyte” or “Th lymphocyte”. MHC Class II molecules present antigen to CD4⁺ Th cells and once activated, Th cells contribute to the activation of CTLs and B lymphocytes via physical contact and cytokine release.

As used herein, “cytotoxicity” or “induce the killing” of an infected cell or hyperproliferative cell, e.g. neoplastic cell, e.g. benign hyperplastic cell, refers to the partial or complete elimination of such cells by a CD8⁺ T cell (or CTL), and does not necessarily indicate a total elimination of the infection or neoplastic growth.

The term “cytokine” is meant to include any one of the group of hormone-like mediators produced by T and B lymphocytes. Representative cytokines include but are not limited to Interleukin-1 (IL-1), IL2, IL3, IL4, IL-5, IL-6, IL-7, IL-8, IL-9, IL-10, IL-11, IL-12, IL-13, IL-15, IL-18, Interferon gamma (IFN-γ), Tumor Necrosis Factor alpha (TNF-α), and Transforming Growth Factor-beta (TGF-β). An “active” fragment of a cytokine is a fragment of a cytokine that retains activity as determined using standard in vitro and in vivo assays. For example, assays for determining IL2 and IFN-γ activity are known in the art (See e.g. Campos, M. (1989) Cell. Immun. 120:259-269 and Czarniecki, C. W. (1986) J. Interferon Res. 6:29-37.) Assays for determining the activity of other cytokines are known and can readily be conducted by those having ordinary skill in the art.

The term “immune response” includes any response associated with immunity including, but not limited to, increases or decreases in cytokine expression, production or secretion (e.g., IL-12, IL-10, TGFβ or TNFα expression, production or secretion), cytotoxicity, immune cell migration, antibody production and/or immune cellular responses. The phrase “modulating an immune response” or “modulation of an immune response” includes upregulation, potentiating, stimulating, enhancing or increasing an immune response, as defined herein. For example, an immune response can be upregulated, enhanced, stimulated or increased directly by use of a modulator of the present invention (e.g., a stimulatory modulator). Alternatively, a modulator can be used to “potentiate” an immune response, for example, by enhancing, stimulating or increasing immune responsiveness to a stimulatory modulator. The phrase “modulating an immune response” or “modulation of an immune response” also includes downregulation, inhibition or decreasing an immune response as defined herein.

Immune responses in a subject or patient can be further characterized as being either type-1 or type-2 immune responses.

A “type-1 immune response”, also referred to herein as a “type-1 response” or a “T helper type 1 (Th1) response” includes a response by CD4⁺ T cells that is characterized by the expression, production or secretion of one or more type-1 cytokines and that is associated with delayed type hypersensitivity responses. The phrase “type-1 cytokine” includes a cytokine that is preferentially or exclusively expressed, produced or secreted by a Th1 cell, that favors development of Th1 cells and/or that potentiates, enhances or otherwise mediates delayed type hypersensitivity reactions. Preferred type-1 cytokines include, but are not limited to, GM-CSF, IL-2, IFN-γ, TNF-α, IL-12, IL-15 and IL-18.

Included within a Th1-mediated response is a CTL-mediated immune response. The term “CTL-mediated immune response” includes any response associated with cytotoxic T cell (CD8⁺ T cell) immunity including, but not limited to, increases or decreases in cytokine expression, production or secretion (e.g., IL-2, IL-12, IL-15, or IFN-γ expression, production or secretion), cytotoxicity, immune cell migration, antibody production and/or immune cellular responses. The phrase “modulating a CTL-mediated immune response” or “modulation of a CTL-mediated immune response” includes upregulation, potentiating, stimulating, enhancing or increasing an immune response, as defined herein. For example, a CTL-mediated immune response can be upregulated, enhanced, stimulated or increased directly by use of an OSPF of the present invention (e.g., a stimulatory modulator). Alternatively, an OSPF can be used to “potentiate” a CTL-mediated immune response, for example, by enhancing, stimulating or increasing immune responsiveness to a stimulatory modulator. The phrase “modulating a CTL-mediated immune response” or “modulation of a CTL-mediated immune response” also includes downregulation, inhibition or decreasing a CTL-mediated immune response as defined herein.

The phrase “type-1 immunity” includes immunity characterized predominantly by type-1 immune responses (e.g., cellular cytotoxicity, delayed type hypersensitivity, and/or macrophage activation), by expression, production or secretion of at least one type-1 cytokine and/or expression of a type-1 immunity cytokine profile. The phrase “potentiating or potentiation of a type-1 or type-2 immune response” includes upregulation, stimulation or enhancement of a type-1 or type-2 response, respectively (e.g., commitment of T helper precursors to either a Th1 or Th2 lineage, further differentiation of cells to either the Th1 or Th2 phenotype and/or continued function of Th1 or Th2 cells during an ongoing immune response). For a review of Th1 and Th2 subsets see, for example, Seder and Paul (1994) Ann. Rev. Immunol. 12:635-673.

A “type-2 immune response”, also referred to herein as a “type-2 response or a “T helper type 2 (Th2) response” refers to a response by CD4⁺ T cells that is characterized by the production of one or more type-2 cytokines and that is associated with humoral or antibody-associated immunity (e.g., efficient B cell, “help” provided by Th2 cells, for example, leading to enhanced modification of certain IgG subtypes and/or IgE). The phrase “type-2 cytokine” includes a cytokine that is preferentially or exclusively expressed, produced or secreted by a Th2 cell, that favors development of Th2 cells and/or that potentiates, enhances or otherwise mediates antibody production by B lymphocytes. Preferred type-2 cytokines include, but are not limited to, IL-4, IL-5, IL-6, IL-10, and IL-13.

As used herein, the term “activity”, “biological activity” or “functional activity”, refers to an activity exerted by a molecule of the invention e.g., an OSPF, as determined in vivo, or in vitro, according to standard techniques and/or methods such as those described in the Examples.

Embodiments of the Invention

The present invention provides, at least in part, methods and compositions for the treatment of immune disorders, such as, for example, viral, bacterial and parasitic infections, prion disease, neoplastic diseases and protection against toxins. The invention is based on the discovery that overlapping synthetic peptide formulations (OSPFs) of the present invention are able to modulate a cytotoxic T lymphocyte (CTL)-mediated response.

Accordingly, the present invention provides a method of modulating, e.g. inducing, an immune response, i.e., a Th1-mediated immune response such as a CTL-mediated immune response or a Th2-mediated immune response and an antibody-associated immune response, by administering to a subject, e.g., a vertebrate, such as a human, an effective amount of an OSPF. The OSPF of the present invention includes a combination, i.e., two or more, of single chain peptides that correspond to an amino acid sequence of a protein of interest, such that the single chain peptide is a length represented by Y, wherein Y is at least 7 to (X-1) and where X is the number of amino acids of the protein of interest, and where at least 1 single chain peptide overlaps with another single chain peptide by a length of Z, wherein Z is 1 to (Y-1), such that the length of the single chain peptide is such that it is able to be internalized by a MHC-bearing cell and can be presented on a MHC molecule to a T cell.

In another embodiment, the OSPF of the present invention includes a combination of single chain peptides that correspond to an amino acid sequence of a protein of interest, such that the single chain peptide is a length represented by Y, wherein Y is at least 7 to (X-1) and where X is the number of amino acids of the protein of interest, such that the length of the single chain peptide is such that it is able to be internalized by a MHC-bearing cell and can be presented on a MHC molecule to a T cell.

In a particular embodiment, Y is at least 7, 8, 9, 10, 11, 12, 13, 14, 15, 16, 17, 18, 19, 20, 21, 22, 23, 24, 25, 26, 27, 28, 29, or 30 amino acids.

In another embodiment, the overlap between single chain peptides is at least 1, 2, 3, 4, 5, 6, 7, 8, 9, 10, 11, 12, 13, 14, 15, 16, 17, 18, 19, 20, 21, 22, 23, 24, 25, 26, 27, 28 or 29 amino acids.

The invention also includes several variations of an OSPF. Examples include, but are not limited to, an OSPF alone or in combination with other proteins or peptides, e.g., a single set of OSPFs from one protein of interest; two or more OSPFs from the same organism or tumor, but different proteins of interest; different OSPFs from different proteins of interest from different organisms or tumors; a single set of OSPFs from a protein of interest and a killed or attenutated organism; a single set of OSPFs and a tumor-related protein (i.e. a tumor antigen); a single set of OSPFs from a protein of interest one or more antibody epitopic peptides; and a single set of OSPFs and one or more Th-related epitopic peptides.

The number of single chain peptides, the length of single chain peptides, and the amount of overlap between single chain peptides will depend on several characteristics of the protein of interest, including the length. These factors can be determined by one skilled in the art without undue experimentation through the use of commercially available computer programs, such as Potean II™ (Proteus) and SPOT™. This allows for several possible epitopes to be encompassed within the OSPF of the present invention, therefore eliminating the cumbersome and expensive step of epitope identification.

In yet another embodiment, the protein of interest includes, but is not limited to, HIV Gag protein (SEQ ID NO:339); SIV Envelope protein (SEQ ID NO:340); anthrax toxins translocating protein (protective antigen precursor [PA]) (SEQ ID NO:209); Ebola virus nucleoprotein (SEQ ID NO:210); hepatitis C virus (HCV) polyprotein (SEQ ID NO:211); melanoma antigen p15 (SEQ ID NO:212); human Her2/neu protein (SEQ ID NO:213); respiratory syncytial virus (RSV) fusion protein (SEQ ID NO:214); HIV-2 gp41 protein (SEQ ID NO:215); HIV-2 GAG protein (SEQ ID NO:216); HIV-2 envelope (env) protein (SEQ ID NO:217); HIV-1 vpu protein (SEQ ID NO:218); HIV-1 envelope (env) protein (SEQ ID NO: 219); HIV-1 Tat interactive protein 2 (SEQ ID NO:220); HIV-1 reverse transcriptase (SEQ ID NO:221) and HIV-1 nef protein (SEQ ID NO:222); circumsporozoite protein precursor (SEQ ID NO:223); circumsporozoite protein II (SEQ ID NO:224); pertussis-like toxin subunit (SEQ ID NO:225); S. aureus enterotoxin A (SEQ ID NO:226); E. coli enterotoxin A (SEQ ID NO:227); C. difficile enterotoxin A (SEQ ID NO:228); B. cereus enterotoxin A (SEQ ID NO:229); pertussis toxin subunit 3 (SEQ ID NO:230)); SARS coronavirus (Frankfurt 1) envelope protein E (SEQ ID No:231); Human metapneumovirus fusion protein (SEQ ID NO:232); SARS coronavirus matrix protein (SEQ ID NO: 233); coronavirus nucleocapsid protein (SEQ ID NO: 234); and SARS coronavirus (Frankfurt 1) spike protein S (SEQ ID NO: 235). For example, OSPFs for HIV-1 Gag include the peptides set forth as SEQ ID NO:1-122 and/or SEQ ID NO:236-335 and OSPFs for SIV Envelope protein include the peptides set forth as SEQ ID NO:123-206 and/or 336-338. For example, OSPFs for HIV-1 Gag include the peptides set forth as SEQ ID NO:1-122 and/or SEQ ID NO:236-335 and OSPFs for SIV Envelope protein include the peptides set forth as SEQ ID NO:123-206 and/or 336-338.

Therapeutic Methods

The present invention provides for therapeutic methods of treating subjects (e.g., vertebrates, such as humans). In one aspect, the invention pertains to a method of treating an OSPF-associated disorder, e.g., any disease, disorder, or condition which can be treated or prevented by modulating an immune response, i.e., a Th1-mediated immune response such as a CTL-mediated immune response or a Th2-mediated immune response, such as an antibody-associated response, in a subject. In one embodiment, the present invention includes administering to a subject having an OSPF-associated disorder, an effective amount of an OSPF of the present invention, thereby treating the OSPF-associated disorder in the subject.

Also within the scope of this invention is the administration of an OSPF prophylactically. Administration of an OSPF of the present invention can occur prior to the manifestation of symptoms of an OSPF-associated disorder, such that the disorder is prevented or, alternatively, delayed in its progression. The prophylactic methods of the present invention can be carried out in a similar manner to the therapeutic methods described herein, although dosage and treatment regimens may differ.

Accordingly, the present method has therapeutic utility in modulating an immune response. In one embodiment, the present method has therapeutic utility in biasing an immune response towards a Th1-mediated (i.e., CTL-mediated) immune response depending upon the desired therapeutic regimen. In another embodiment, the present invention has therapeutic utility in biasing an immune response towards a Th2-mediated (i.e., antibody-associated immunity). Such methods are particularly useful in diseases such as viral infections (e.g., Ebola virus, hepatitis C, HIV, e.g., HIV-1 and HIV-2, RSV, monkeypox, and SARS coronavirus), bacterial infections (e.g., anthrax, Listeria monocytogenes, Legionella and mycobacterium such as tuberculosis), parasitic infections (e.g. malaria) protection against toxins (e.g., shigella toxin, toxin botulinum and tetanus toxin), prion diseases, and neoplastic diseases (e.g., breast, colon, non-small cell lung, head and neck, colorectal, lung, prostate, ovary, renal, melanoma, gastrointestinal (e.g., pancreatic and stomach) cancer and osteogenic sarcoma).

In another aspect, the invention provides a vaccine for immunizing a subject against an OSPF-associated disorder, wherein the vaccine comprises an OSPF of the present invention, either alone or dispersed in a physiologically acceptable, nontoxic vehicle in an amount is effective to immunize a subject against an OSPF disorder.

The vaccines of the present invention are administered in a manner compatible with the dosage formulation, and in such amount as will be therapeutically effective and immunogenic. The quantity to be administered depends on the subject to be treated, capacity of the subject's immune system to generate a cellular immune response, and degree of protection desired. Precise amounts of active ingredient required to be administered depend on the judgment of the practitioner and are peculiar to each individual. However, suitable dosage ranges are of the order of about one microgram to about one milligram, preferably about 25 micrograms and more preferably about 30 micrograms active ingredient per kilogram per 70 kilogram individual. Suitable regimes for initial administration and booster shots are also variable, but are typified by an initial administration followed in one or two week intervals by a subsequent injection or other administration. Also within the scope of the invention is the co-administration of an adjuvant in combination with an OSPF of the present invention. Suitable adjuvants include, but are not limited to, IL-2, IL-12, IL-15, alum, Conconvalin A, phorbol esters and Freud's adjuvant.

In yet another aspect, the invention features a kit for immunizing a subject against an OSPF-associated disorder wherein the kit comprises an OSPF of the present invention and may further comprise instructions for use.

In yet another aspect, the invention features a vaccine adjuvant which comprises an OSPF of the present invention and a pharmaceutically acceptable carrier which may be used to enhance the efficacy of a vaccine.

Pharmaceutical Compositions and Uses thereof

Another aspect of the present invention provides pharmaceutically-acceptable compositions which comprise an OSPF and a pharmaceutically-acceptable carrier(s), in an amount effective to modulate a CTL-mediated immune response.

In a particular embodiment, the OSPF is administered to the subject using a pharmaceutically-acceptable formulation, e.g., a pharmaceutically-acceptable formulation that provides sustained delivery of the OSPF to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

As used herein, the term “effective amount” includes an amount effective, at dosages and for periods of time necessary, to achieve the desired result, e.g., sufficient to modulate a CTL-mediated immune response. An effective amount of OSPF, as defined herein may vary according to factors such as the disease state, age, and weight of the subject, and the ability of the OSPF to elicit a desired response in the subject. Dosage regimens may be adjusted to provide the optimum therapeutic response. An effective amount is also one in which any toxic or detrimental effects (e.g., side effects) of the OSPF of the present invention are outweighed by the therapeutically beneficial effects.

A therapeutically effective amount of OSPF (i.e., an effective dosage) may range from about 0.001 to 40 μg/kg body weight, preferably about 0.01 to 30 μg/kg body weight per 70 kilogram individual. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an OSPF can include a single treatment or, can include a series of treatments. In one example, a subject is treated with an OSPF in the range of between about 0.1 to 30 μg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of an OSPF used for treatment may increase or decrease over the course of a particular treatment.

The methods of the invention further include administering to a subject a therapeutically effective amount of an OSPF in combination with another pharmaceutically active compound known to modulate, for example, a CTL-mediated immune responses, e.g., agents such as interleukins (IL) (e.g. IL-2, IL-12, IL-15), lipopolysaccharide (LPS), concanavalin A (ConA), phorbol esters, and ionomycin. Other pharmaceutically active compounds that may be used to modulate a TH2-mediated immune response, for example, can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference. The OSPF and the pharmaceutically active compound may be administered to the subject in the same pharmaceutical composition or in different pharmaceutical compositions (at the same time or at different times).

The regimen of administration also can affect what constitutes an effective amount. OSPFs of the present invention can be administered to the subject prior to, simultaneously with, or after the administration of the other agent(s). Further, several divided dosages, as well as staggered dosages, can be administered daily or sequentially, or the dose can be proportionally increased or decreased as indicated by the exigencies of the therapeutic situation.

The phrase “pharmaceutically acceptable” is employed herein to refer to those OSPFs of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (1₃) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing an OSPF(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these compositions include the step of bringing into association an OSPF(s) with the carrier and, optionally, one or more accessory ingredients. In general, the formulations are prepared by uniformly and intimately bringing into association an OSPF with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an OSPF(s) as an active ingredient. An OSPF may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the OSPF(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active OSPF(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more OSPF(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of an OSPF(s) include powders, sprays, ointments, pastes, creams, lotions, gels, solutions, patches and inhalants. The active OSPF(s) may be mixed under sterile conditions with a pharmaceutically-acceptable carrier, and with any preservatives, buffers, or propellants which may be required.

The ointments, pastes, creams and gels may contain, in addition to OSPF(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an OSPF(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The OSPF(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of an OSPF(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of this invention.

Pharmaceutical compositions of this invention suitable for parenteral administration comprise one or more OSPF(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form.

Alternatively, delayed absorption of a parenterally-administered drug form is accomplished by dissolving or suspending the drug in an oil vehicle.

Injectable depot forms are made by forming microencapsule matrices of OSPF(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the OSPF(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

The term “administration” or “administering” is intended to include routes of introducing the OSPF(s) to a subject to perform their intended function. Examples of routes of administration which can be used include injection (subcutaneous, intravenous, parenterally, intraperitoneally, intrathecal), oral, inhalation, rectal and transdermal. The pharmaceutical preparations are, of course, given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the OSPF can be coated with or disposed in a selected material to protect it from natural conditions which may detrimentally effecting its ability to perform its intended function. The OSPF can be administered alone, or in conjunction with either another agent as described above or with a pharmaceutically-acceptable carrier, or both. The OSPF can be administered prior to the administration of the other agent, simultaneously with the agent, or after the administration of the agent. Furthermore, the OSPF can also be administered in a proform which is converted into its active metabolite, or more active metabolite in vivo.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically”, “peripheral administration” and “administered peripherally” as used herein mean the administration of an OSPF(s), drug or other material, such that it enters the subject's system and, thus, is subject to metabolism and other like processes, for example, subcutaneous administration.

Regardless of the route of administration selected, the OSPF(s), which may be used in a suitable hydrated form, and/or the pharmaceutical compositions of the present invention, are formulated into pharmaceutically-acceptable dosage forms by conventional methods known to those of skill in the art.

### EXAMPLES

The invention is further illustrated by the following examples which in no way should be construed as being further limiting.

### 1. Example 1

### HIV and SIV

Materials and Methods

A. Peptides

OSPFs corresponding to HIV Gag (SEQ ID NOs:1-122) represent a group of peptides of 15 amino acids in length, with 11-amino acid overlaps between sequential peptides, and spanning the entire HIV Gag protein. Most peptides were approximately 80% pure. OSPFs corresponding to SIV Env (SEQ ID NOs:123-206 and/or 336-338), represent a group of peptides of 20 amino acids in length, with 10 amino acid overlaps between sequential peptides, and spanning the entire SIV Env protein. Most peptides are approximately 80% pure.

Peptide P7G (AMQMLKETI (SEQ ID NO:207)) is an H-2K^{d}-restricted CTL epitope of HIV p24 antigen (see, e.g., Doe, B and Walker, C. (1996) AIDS 10:793). This peptide was made by the Molecular Biology Core Facilities at the Dana-Farber Cancer Institute (DFCI), and was used as a positive control. The peptide was greater than approximately 97% pure.

A non-epitope peptide, HIV clade C envelope V3 peptide (GPGQAFYAT (SEQ ID NO:208) made by the Molecular Biology Core Facilities, at Dana Farber Cancer Institute, Boston, Mass., was used as a negative control peptide. The peptide was approximately 97% pure.

B. Mice and Immunization

BALB/c (H-2^{d}) and C57BL/6 (H-2^{b}) (Taconic Farms, N.Y.), were immunized subcutaneously (s.c.) with OSPF-HIV Gag. Each mouse was immunized with 5 μg of each peptide, combined with MLP+TDM Adjuvant System (Sigma, St. Louis, Mo.; product number M6536. Peptides were >80% pure.). The HIV Gag OSPF was a series of peptides, each 15 amino acids in length, with 11-amino acid overlaps between sequential peptide (NIH AIDS Research and Reference Reagent Program Catalog #5107). Control mice were given adjuvant alone (mock immunization). The immunization regimen is shown below in Table 1:

TABLE 1

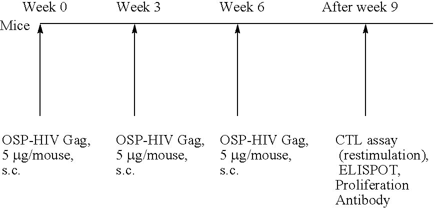

C. Blood Donors and Isolation and Differentiation of Blood Dendritic Cells

Leukopacks were provided by anonymous, normal blood donors (Dana-Farber Cancer Institute Blood Bank, Boston, Mass.). These donors were MHC tissue-typed in Brigham and Women's Hospital (Boston, Mass.) and shown to have different MHC antigens. Dendritic cells (DC) were isolated and differentiated from peripheral blood mononuclear cells (PBMC). PBMC were cultured in plastic cell-culture flasks and incubated for 2 hrs at 5% CO₂ and 37° C. The adherent cells were collected and incubated in complete RPMI supplemented with interleukin-4 (IL-4) and granulocyte-macrophage colony stimulating factor (GM-CSF) (Stem Cell Technology, Vancouver, Canada) (DC medium). An additional 2 ml of DC medium was added to the culture each day. On day 6, detached cells were collected and transferred into a new flask with fresh DC medium. The purity of the DC cell population was assessed using monoclonal antibodies against specific DC markers (see, e.g., Popov, S., unpublished data). These DC were pulsed overnight with OSPF-SIV Env. The OSPF-SIV Env are a series of peptides of 20 amino acids in length, with 10-amino acid overlaps between sequential peptides (NIH AIDS Research and Reference Reagent Program Catalog #4625). Peptides were >80% pure. DC were irradiated and used to generate CTL in vitro by 3 stimulation of autologous PBMC at weekly interval. A CTL assay or ELISPOT was performed one week after the last stimulation.

D. Cytotoxic T Lymphocyte (CTL) Assays

Murine CTL Assays:

For the mouse CTL assay, effector cells were splenic mononuclear cells which were isolated from OSPF- or adjuvant-only immunized mice and restimulated (2×10⁶/ml) in vitro with 1 μM peptide for 7-10 days. Target cells were P815 cells (H-2^{d}, for BALB/c mice) and EL-4 cells (H-2^{b}, for B57BL/6 mice). Target cells were labeled with ⁵¹Cr (70 μCi/2×10⁶ cells; Perkin-Elmer, Boston, Mass.) and pulsed overnight with or without OSPF-HIV Gag (1 μM), or infected overnight with vaccinia virus [2 plaque forming unit (pfu)/target cell] expressing HIVgag (NIH AIDS Research and Reference Reagent Program cat # vP1289), or wild type vaccinia virus (Therion, Cambridge, Mass.).

In the case of H-2^{d} restricted CTL, a known CTL epitope from HIV p24 antigen P7G (AMQMLKETI)¹⁹ (SEQ ID NO:207) (>97% pure, Molecular Biology Core Facilities, Dana farer Cancer Institute, Boston, Mass.) was included to test if OSPF-HIV Gag could generate P7G specific (H-2^{d} restricted) CTL in BALB/c mice. A non-epitopic peptide, HIV clade C envelope V3 peptide (GPGQAFYAT) (SEQ ID NO:208), (>97% pure, Molecular Biology Core Facilities, Dana Farber Cancer Institute, Boston, Mass.), was used as negative control peptide.

Effector cells and target cells were co-cultured at different ratios for 6 h, and cytolysis was determined by ⁵¹Cr release from target cells (see, e.g., Wunderlich et al., (1997) Current Protocols in Immunology 3.11.1-3.11.20). The percentage specific ⁵¹Cr release was calculated as: 100 (experimental release−spontaneous release)/(maximum release−spontaneous release). Maximum release was determined from supernatants of cells that were lysed by addition of 5% Triton-X 100. Spontaneous release was determined from the target cells incubated without addition of effector cells.

Human CTL Assays:

For the human CTL assay, effector cells were PBMC stimulated with irradiated autologous DC that had been pulsed with or without OSPF cells (see Table 2). Target cells were EBV-transformed, autologous B cell lines. These cells were labeled with ⁵¹Cr (70 μCi/2×10⁶ cells; Perkin-Elmer, Boston, Mass.) and pulsed overnight with or without OSPF-SIV Env (1 μM), or infected overnight with vaccinia virus [2 plaque forming unit (pfu)/target cell] expressing SIV gag-pol-env, or wild type vaccinia virus (Vaccinia virus expressing SIV gag-pol-env and wild type vaccinia virus were obtained from Therion, Cambridge, Mass.).

Effector cells and target cells were co-cultured and the percentage specific ⁵¹Cr release was calculated as described above in the mouse CTL assay section (see, e.g., Wunderlich, et al., supra).

TABLE 2

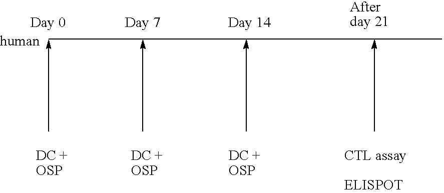

E. ELISPOT™ Assay

Human and mouse ELISPOT assays were performed using ELISPOT kits from BioSource International (Camarillo, Calif.). Briefly, following the final stimulation, mouse splenocytes or human PBMC stimulated with DC (treated with OSPF and untreated) were seeded into anti-interferon gamma (anti-IFN-γ) monoclonal antibody coated 96-well plates and incubated overnight at 4° C. Subsequently, the cells were discarded and biotinated- anti-IFN-γ antibodies were added for an hour at 37° C. followed by another hour of incubation at 37° C. with anti-biotin antibody labeled with enzyme. After the color reaction developed, spots were counted under a microscope. Results were expressed as spot forming units (SFU)/10⁶ cells.

F. Lymphocyte Proliferation Assay

Splenic lymphocytes were isolated and cultured at 2×10⁶/ml in RPMI 1640 plus 15 % FCS and antibiotics in the presence of HIV Gag protein (15 ug/ml), OSPF-HIV Gag (3 ug/ml) or ovalbumin (OVA) (15 ug/ml) for 5 days. Four hours prior to harvesting, cells were pulsed with 1 uCI per well of ³H-thymidine. After cells were harvested, ³H-thymidine incorporation was assessed using a β-counter (Beckman). Results were expressed as count per minute (cpm).

Results

A. OSPF-HIV Gag can Promiscuously Induce CTL Responses in Genetically Different Mice

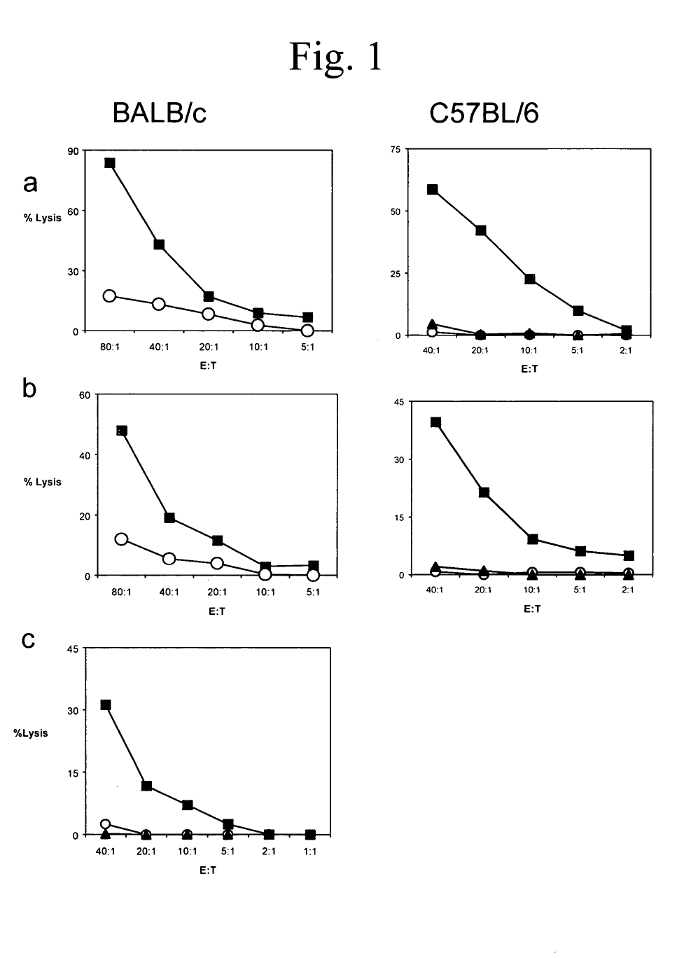

To determine whether OSPF were able to induce CTL responses in genetically different mice, BALB/c (H-2^{d}) and C57BL/6 (H-2^{b}) mice were immunized subcutaneously three times at three-week intervals with OSPF-HIV Gag together with an oil-in-water adjuvant system MPL+TDM. CTL activity in both mouse strains against OSPF-HIV Gag was detected by ⁵¹Cr release assays (FIG. 1a). No CTL activity was detected in the control mice (adjuvant only). Moreover, these CTLs were also capable of killing target cells infected with vaccinia virus engineered to express HIV Gag (FIG. 1b), and, in the case of BALB/c mice, and HIV Gag specific, H-2K^{d} restricted epitope P7G (FIG. 1c). These results suggest that not only are OSPF-HIV Gag able to generate specific CTLs, but these cells are capable of killing cells which express HIV-Gag protein.

B. OSPF-HIV Gag can Induce Proliferative Th Cell Responses in Genetically Different Mice

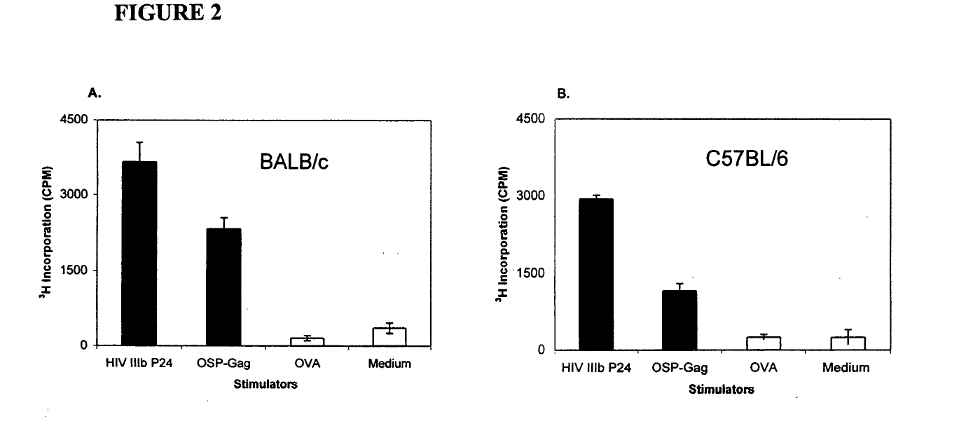

To determine whether OSPF are capable of stimulating a proliferative Th cell response, BALB/c and C57BL/6 were immunized with OSPF-HIV Gag as described above. Splenocytes were recovered and cultured in vitro with either soluble HIV Gag protein, OSPF-HIV Gag or ovalbumin as a control. The proliferative response was measured by the percentage of ³H-thymidine incorporation (FIG. 2). These results demonstrate that OSPF can induce a proliferative Th response and that immunizing with OSPF provides the same proliferative Th-mediated response as does that of the intact protein.

C. Ex vivo Induction of Dendritic Cells and Autologous PBMCs of Human Individuals with Different MHC Class I Backgrounds

OSPF that corresponded to SIV Env (OSPF-SIV Env) were used to induce the virus-specific CTL responses ex vivo using cells from human blood leukopacks (dendritic cells (DC) and autologous PBMC). OSPF-SIV Env are a group of 87 peptides of 20 amino acids in length, with 10 amino acid overlaps between sequential peptides, and spanning the entire SIV Env protein OSP promiscuously induced CTL in different individuals of different MHC backgrounds.

Cells from two human blood leukopacks from two anonymous donors (d#1 and d#2) were collected and their MHC class I (HLA—A, B, C) were tested [d#1: HLA-A (02, blank); B (08, 18); Bw4 (−,−); Bw6 (+,+); Cw(07, blank). D#2: HLA-A (11, 24); B (39, 51); Bw4 (−,+); Bw6 (+,−); Cw (07, 14). The peripheral blood monocytes (PBMC) were separated and stimulated three times in vitro with irradiated autologous dendritic cells (DC) pulsed with or without OSPF-SIV Env at weekly intervals and ELISPOT and chromium release assays were performed one week after the last stimulation.

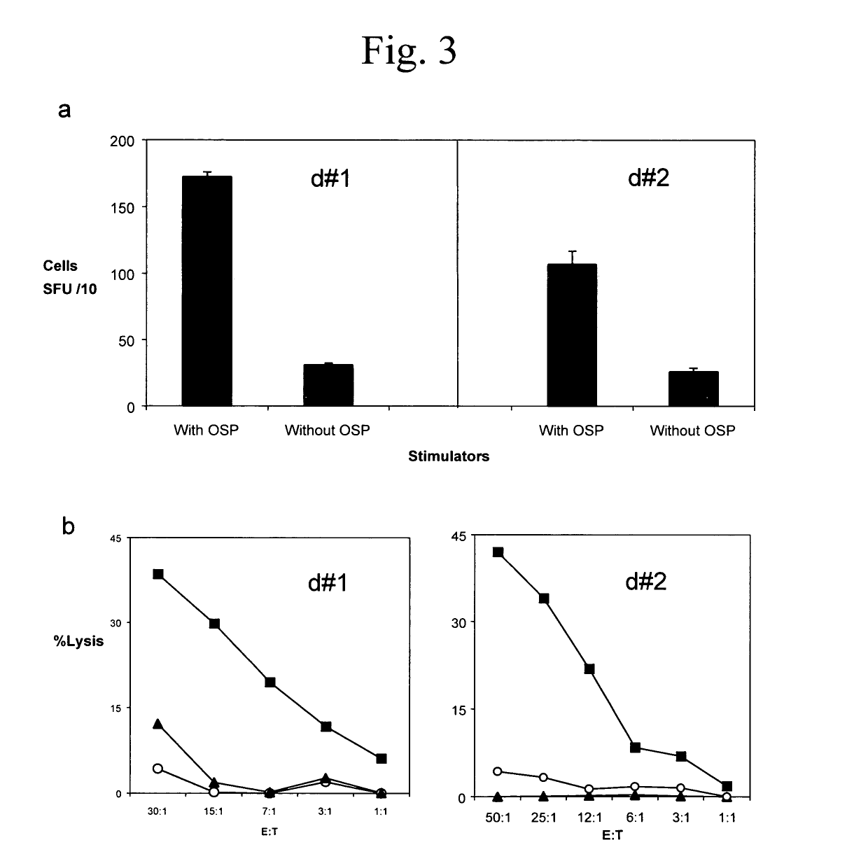

These results show that PBMC stimulated with DC pulsed with OSPF-SIV Env generated interferon-y secreted cells in both d#1 and d#2 (FIG. 3a). The chromium release assay showed that target cells transfected with vaccinia virus expressing SIV gag-pol-env were also killed by CTL (FIG. 3b). There was no killing when effector and target cells from two leukopacks were mismatched (data not shown), indicating that the APC from the two leukopacks did not present the same epitopes and the killing was MHC restricted.

Conclusions

These results show that an individual OSPF can generate CTL activity and proliferative Th cell mediated responses in genetically different strains of mice. Furthermore, OSPF can generate CTL activity in human cells with different HLA subtypes. The data also shows that immunization with OSPF(s) can result in the generation of antigen-specific CTL cells capable of lysing virally-infected cells (i.e. cells pulsed with OSPF, target cells infected with vaccinia expressing HIV genes and target cells pulsed with virus-specific epitopic peptide P7G (AMQMLKETI) (SEQ ID NO:207). Thus, since OSPF(s) are capable of generating a Th proliferative response and CTLs in genetically diverse individuals/animals, there is no need to identify specific CTL epitopes.

### 2. Example 2

### RSV

A. Materials and Methods

Potential OSPFs corresponding to RSV fusion protein (SEQ ID NO:214) are shown as follows (The numbered and underlined sequences represent the single chain peptide sequences):

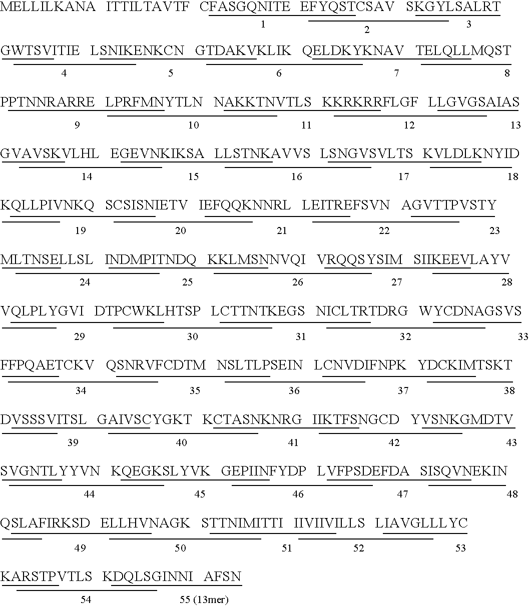

The OSPFs corresponding to the RSV fusion protein represent a group of 55 peptides of 15 amino acids in length, with 5 amino acid overlaps between sequential peptides, and spanning the entire RSV fusion protein.

Examples of other proteins of interest which can be used in the present invention, include, but are not limited to, anthrax toxins translocating protein (protective antigen precursor [PA]) (SEQ ID NO:209); Ebola virus nucleoprotein (SEQ ID NO:210); hepatitis C virus (HCV) polyprotein (SEQ ID NO:211); melanoma antigen p15 (SEQ ID NO:212); human Her2/neu protein (SEQ ID NO:213); HIV-2 gp41 protein (SEQ ID NO:215); HIV-2 GAG protein (SEQ ID NO:216); HIV-2 envelope (env) protein (SEQ ID NO:217); HIV-1 vpu protein (SEQ ID NO:218); HIV-1 envelope (env) protein (SEQ ID NO: 219); HIV-1 Tat interactive protein 2 (SEQ ID NO:220); HIV-1 reverse transcriptase (SEQ ID NO:221) and HIV-1 nef protein (SEQ ID NO:222); circumsporozoite protein precursor (SEQ ID NO:223); circumsporozoite protein II (SEQ ID NO:224); pertussis-like toxin subunit (SEQ ID NO:225); S. aureus enterotoxin A (SEQ ID NO:226); E. coli enterotoxin A (SEQ ID NO:227); C. difficile enterotoxin A (SEQ ID NO:228); B. cereus enterotoxin A (SEQ ID NO:229); pertussis toxin subunit 3 (SEQ ID NO:230)); SARS coronavirus (Frankfurt 1) envelope protein E (SEQ ID No:23 1); Human metapneumovirus fusion protein (SEQ ID NO:232); SARS coronavirus matrix protein (SEQ ID NO: 233); coronavirus nucleocapsid protein (SEQ ID NO: 234); and SARS coronavirus (Frankfurt 1) spike protein S (SEQ ID NO: 235).

B. Vaccinia Viruses

Vaccinia viruses expressing RSV fusion protein may be utilized and can be made using routine techniques known to those skilled in the art to conduct CTL assays in vitro.

C. Mice and Immunization

BALB/c (H-2^{d}) and C57BL/6 (H-2^{b}) are immunized subcutaneously (s.c.) with OSPF of RSV fusion protein at 5 μg of each individual peptide per mouse together with MLP+TDM Adjuvant system (Sigma, St. Louis, Mo.; product number M6536. Peptides were >80% pure). Control mice are given only the adjuvant (mock immunization) according to the regimen described in Example 1.

D. Blood Donors and Isolation and Differentiation of Blood Dendritic Cells

Leukopacks may be provided by anonymous, normal blood donors. These donors are MHC tissue-typed and dendritic cells isolated and differentiated as previously described above in Example 1.

E. Cytotoxic T Lymphocyte (CTL) Assays

Murine CTL Assays:

For the mouse CTL assay, effector cells are splenic mononuclear cells which are isolated from OSPF- or adjuvant-only immunized mice and restimulated (2×10⁶/ml) in vitro with 1 μM peptide for 7-10 days. Target cells are P815 cells (H-2^{d}, for BALB/c mice) and EL-4 cells (H-2^{b}, for B57BL/6 mice). Target cells are labeled with ⁵¹Cr (70 μCi/2×10⁶ cells; Perkin-Elmer, Boston, Mass.) and pulsed overnight with or without OSPF-RSV fusion protein (1 μM), or infected overnight with vaccinia virus [2 plaque forming unit (pfu)/target cell].

Effector cells and target cells are co-cultured at different ratios for 6 h, and cytolysis is determined by ⁵¹Cr release from target cells (see, e.g., Wunderlich et al., (1997) Current Protocols in Immunology 3.11.1-3.11.20). The percentage specific ⁵¹Cr release is calculated as: 100 (experimental release−spontaneous release)/(maximum release−spontaneous release). Maximum release is determined from supernatants of cells that are lysed by addition of 5% Triton-X 100. Spontaneous release is determined from the target cells incubated without addition of effector cells.

Human CTL Assays:

For the human CTL assay, effector cells are PBMC stimulated with irradiated 20 autologous DC that are pulsed with or without OSPF (see Table 2).Target cells are EBV-transformed, autologous B cell lines. These cells are labeled with ⁵¹Cr (70 μCi/2×10⁶ cells; Perkin-Elmer, Boston, Mass.) and pulsed overnight with or without OSPF RSV fusion protein (1 μM), or infected overnight with vaccinia virus [2 plaque forming unit (pfu)/target cell] expressing SIV gag-pol-env, or wild type vaccinia virus (Vaccinia virus expressing SIV gag-pol-env and wild type vaccinia virus are obtained from Therion, Cambridge, Mass.).

Effector cells and target cells are co cultured and the percentage specific ⁵¹Cr release is calculated as described above in the mouse CTL assay section (see, e.g., Wunderlich, et al., supra).

F. ELISPOT™ Assay

Human and mouse ELISPOT assays are performed using ELISPOT kits from BioSource International (Camarillo, Calif.). Briefly, following the final stimulation, mouse splenocytes or human PBMC are stimulated with DC (treated with OSPF and untreated) and seeded into anti-interferon gamma (anti-IFN-γ) monoclonal antibody coated 96-well plates and incubated overnight at 4° C. Subsequently, the cells are discarded and biotinated- anti-IFN-γ antibodies are added for an hour at 37° C. followed by another hour of incubation at 37° C. with anti-biotin antibody labeled with enzyme. After the color reaction develops, spots are counted under a microscope. Results are expressed as spot forming units (SFU)/10⁶ cells.

G. Lymphocyte Proliferation Assay

Splenic lymphocytes are isolated and cultured at 2×10⁶/ml in RPMI 1640 plus 15% FCS plus antibiotics in the presence of RSV fusion protein (15 ug/ml) or OSPF-RSV fusion protein or ovalbumin (OVA) for 5 days. Four hours before harvesting, cells are pulsed with 1 uCI per well of 3H-thymidine. After cells are harvested, ³H-thymidine incorporation is assessed using a β-counter (Beckman). Results are expressed as count per minute (cpm).

Incorporation by Reference

The contents of all references (including literature references, issued patents, published patent applications, and co-pending patent applications) cited throughout this application are hereby expressly incorporated herein in their entireties by reference.

Equivalents

Those skilled in the art will recognize, or be able to ascertain using no more than routine experimentation, many equivalents of the specific embodiments of the invention described herein. Such equivalents are intended to be encompassed by the following claims.

1. A method of modulating an immune response comprising administering to a subject an effective amount of an overlapping synthetic peptide formulation (OSPF), wherein said OSPF comprises a combination of single chain peptides corresponding to an amino acid sequence of a protein of interest, wherein said single chain peptide is a length represented by Y, wherein Y is at least 7 to (X-1) and X is the number of amino acids of said protein of interest, wherein at least one single chain peptide overlaps with another single chain peptide by a length represented by Z, wherein Z is 1 to (Y-1), wherein said length of said single chain peptide is such that internalization of said single chain peptide by a MHC-bearing cell and presentation by a MHC molecule to a T cell is possible, such that said immune response is modulated.

2. The method of claim 1, wherein said subject is a vertebrate.

3. The method of claim 1, wherein said Y is fifteen (15) amino acids.

4. The method of claim 1, wherein said Z is five (5) amino acids.

5. The method of claim 1, wherein said immune response is a Th1-mediated immune response.

6. The method of claim 5, wherein said Th1-mediated immune response is a CTL-mediated immune response.

7. The method of claim 1, wherein said immune response is a Th2-mediated immune response.

8. The method of claim 7, wherein said Th2-mediated immune response is an antibody-associated immune response.

9. The method of claim 1, wherein said MHC-bearing cell is a MHC Class I-bearing cell.

10. The method of claim 9, wherein said MHC Class I-bearing cell is a CTL.

11. The method of claim 1, wherein said MHC-bearing cell is a MHC Class II-bearing cell.

12. The method of claim 11, wherein said MHC Class II-bearing cell is a B cell.

13. The method of claim 1, wherein said protein of interest is selected from the group consisting of HIV Gag protein (SEQ ID NO:339); SIV Envelope protein (SEQ ID NO:340); anthrax toxins translocating protein (protective antigen precursor [PA]) (SEQ ID NO:209); Ebola virus nucleoprotein (SEQ ID NO:210); hepatitis C virus (HCV) polyprotein (SEQ ID NO:211); melanoma antigen p15 (SEQ ID NO:212); human Her2/neu protein (SEQ ID NO:213); respiratory syncytial virus (RSV) fusion protein (SEQ ID NO:214); HIV-2 gp41 protein (SEQ ID NO:215); HIV-2 GAG protein (SEQ ID NO:216); HIV-2 envelope (env) protein (SEQ ID NO:217); HIV-1 vpu protein (SEQ ID NO:218); HIV-1 envelope (env) protein (SEQ ID NO: 219); HIV-1 Tat interactive protein 2 (SEQ ID NO:220); HIV-1 reverse transcriptase (SEQ ID NO:221) and HIV-1 nef protein (SEQ ID NO:222); circumsporozoite protein precursor (SEQ ID NO:223); circumsporozoite protein II (SEQ ID NO:224); pertussis-like toxin subunit (SEQ ID NO:225); S. aureus enterotoxin A (SEQ ID NO:226); E. coli enterotoxin A (SEQ ID NO:227); C. difficile enterotoxin A (SEQ ID NO:228); B. cereus enterotoxin A (SEQ ID NO:229); pertussis toxin subunit 3 (SEQ ID NO:230)); SARS coronavirus (Frankfurt 1) envelope protein E (SEQ ID No:231); Human metapneumovirus fusion protein (SEQ ID NO:232); SARS coronavirus matrix protein (SEQ ID NO: 233); coronavirus nucleocapsid protein (SEQ ID NO: 234); and SARS coronavirus (Frankfurt 1) spike protein S (SEQ ID NO: 235).

14-20. (canceled)

21. The method of claim 13, wherein said MHC-bearing cell is a MHC Class I-bearing cell.

22. The method of claim 21, wherein said MHC Class I-bearing cell is a CTL.

23-25. (canceled)

26. A pharmaceutical composition comprising an overlapping synthetic peptide formulation (OSPF) and a pharmaceutically acceptable carrier, wherein said OSPF comprises a combination of single chain peptides corresponding to an amino acid sequence of a protein of interest, wherein said single chain peptide is a length represented by Y, wherein Y is at least 7 to (X-1) and X is the number of amino acids of said protein of interest, wherein at least one single chain peptide overlaps with another single chain peptide by a length represented by Z, wherein Z is 1 to (Y-1), wherein said length of said single chain peptide is such that internalization of said single chain peptide by a MHC-bearing cell and presentation by a MHC molecule to a T cell is possible.

27. The pharmaceutical composition of claim 26, wherein said Y is fifteen (15) amino acids.

28. The pharmaceutical composition of claim 27, wherein said Z is five (5) amino acids.

29. The pharmaceutical composition of claim 26, wherein said MHC-bearing cell is a MHC Class I-bearing cell.

30. The pharmaceutical composition of claim 29, wherein said MHC Class I-bearing cell is a CTL.

31. The pharmaceutical composition of claim 26, wherein said MHC-bearing cell is a MHC Class II-bearing cell.

32. The pharmaceutical composition of claim 31, wherein said MHC Class II-bearing cell is a B cell.

33. The pharmaceutical composition of claim 26, wherein said protein of interest is selected from the group consisting of HIV Gag protein (SEQ ID NO:339); SIV Envelope protein (SEQ ID NO:340); anthrax toxins translocating protein (protective antigen precursor [PA]) (SEQ ID NO:209); Ebola virus nucleoprotein (SEQ ID NO:210); hepatitis C virus (HCV) polyprotein (SEQ ID NO:21 1); melanoma antigen p15 (SEQ ID NO:212); human Her2/neu protein (SEQ ID NO:213); respiratory syncytial virus (RSV) fusion protein (SEQ ID NO:214); HIV-2 gp41 protein (SEQ ID NO:215); HIV-2 GAG protein (SEQ ID NO:216); HIV-2 envelope (env) protein (SEQ ID NO:217); HIV-1 vpu protein (SEQ ID NO:218); HIV-1 envelope (env) protein (SEQ ID NO: 219); HIV-1 Tat interactive protein 2 (SEQ ID NO:220); HIV-1 reverse transcriptase (SEQ ID NO:221) and HIV-1 nef protein (SEQ ID NO:222); circumsporozoite protein precursor (SEQ ID NO:223); circumsporozoite protein II (SEQ ID NO:224); pertussis-like toxin subunit (SEQ ID NO:225); S. aureus enterotoxin A (SEQ ID NO:226); E. coli enterotoxin A (SEQ ID NO:227); C. difficile enterotoxin A (SEQ ID NO:228); B. cereus enterotoxin A (SEQ ID NO:229); pertussis toxin subunit 3 (SEQ ID NO:230)); SARS coronavirus (Frankfurt 1) envelope protein E (SEQ ID No:231); Human metapneumovirus fusion protein (SEQ ID NO:232); SARS coronavirus matrix protein (SEQ ID NO: 233); coronavirus nucleocapsid protein (SEQ ID NO: 234); and SARS coronavirus (Frankfurt 1) spike protein S (SEQ ID NO: 235).

34-212. (canceled)

213. The method of claim 1, further comprising an adjuvant.

214-253. (canceled)


SEQUENCE LISTING (340 sequences)

SEQ ID NO: 1 - protein, 15 aa, Human Immunodeficiency Virus
MGARASVLSGGELDR

SEQ ID NO: 2 - protein, 15 aa, Human Immunodeficiency Virus
ASVLSGGELDRWEKI

SEQ ID NO: 3 - protein, 15 aa, Human Immunodeficiency Virus
SGGELDRWEKIRLRP

SEQ ID NO: 4 - protein, 15 aa, Human Immunodeficiency Virus
LDRWEKIRLRPGGKK

SEQ ID NO: 5 - protein, 15 aa, Human Immunodeficiency Virus
EKIRLRPGGKKKYKL

SEQ ID NO: 6 - protein, 15 aa, Human Immunodeficiency Virus
LRPGGKKKYKLKHIV

SEQ ID NO: 7 - protein, 15 aa, Human Immunodeficiency Virus
GKKKYKLKHIVWASR

SEQ ID NO: 8 - protein, 15 aa, Human Immunodeficiency Virus
YKLKHIVWASRELER

SEQ ID NO: 9 - protein, 15 aa, Human Immunodeficiency Virus
HIVWASRELERFAVN

SEQ ID NO: 10 - protein, 15 aa, Human Immunodeficiency Virus
ASRELERFAVNPGLL

SEQ ID NO: 11 - protein, 15 aa, Human Immunodeficiency Virus
LERFAVNPGLLETSE

SEQ ID NO: 12 - protein, 15 aa, Human Immunodeficiency Virus
AVNPGLLETSEGCRQ

SEQ ID NO: 13 - protein, 15 aa, Human Immunodeficiency Virus
GLLETSEGCRQILGQ

SEQ ID NO: 14 - protein, 15 aa, Human Immunodeficiency Virus
TSEGCRQILGQLQPS

SEQ ID NO: 15 - protein, 15 aa, Human Immunodeficiency Virus
CRQILGQLQPSLQTG

SEQ ID NO: 16 - protein, 15 aa, Human Immunodeficiency Virus
LGQLQPSLQTGSEEL

SEQ ID NO: 17 - protein, 15 aa, Human Immunodeficiency Virus
QPSLQTGSEELRSLY

SEQ ID NO: 18 - protein, 15 aa, Human Immunodeficiency Virus
QTGSEELRSLYNTVA

SEQ ID NO: 19 - protein, 15 aa, Human Immunodeficiency Virus
EELRSLYNTVATLYC

SEQ ID NO: 20 - protein, 15 aa, Human Immunodeficiency Virus
SLYNTVATLYCVHQR

SEQ ID NO: 21 - protein, 15 aa, Human Immunodeficiency Virus
TVATLYCVHQRIEIK

SEQ ID NO: 22 - protein, 15 aa, Human Immunodeficiency Virus
LYCVHQRIEIKDTKE

SEQ ID NO: 23 - protein, 15 aa, Human Immunodeficiency Virus
HQRIEIKDTKEALDK

SEQ ID NO: 24 - protein, 15 aa, Human Immunodeficiency Virus
EIKDTLEALDKIEEE

SEQ ID NO: 25 - protein, 15 aa, Human Immunodeficiency Virus
TKEALDKIEEEQNKS

SEQ ID NO: 26 - protein, 15 aa, Human Immunodeficiency Virus
LDKIEEEQNKSKKKA

SEQ ID NO: 27 - protein, 15 aa, Human Immunodeficiency Virus
EEEQNKSKKKAQQAA

SEQ ID NO: 28 - protein, 15 aa, Human Immunodeficiency Virus
NKSKKKAQQAAADTG

SEQ ID NO: 29 - protein, 15 aa, Human Immunodeficiency Virus
KKAQQAAADTGHSNQ

SEQ ID NO: 30 - protein, 15 aa, Human Immunodeficiency Virus
QAAADTGHSNQVSQN

SEQ ID NO: 31 - protein, 15 aa, Human Immunodeficiency Virus
DTGHSNQVSQNYPIV

SEQ ID NO: 32 - protein, 15 aa, Human Immunodeficiency Virus
SNKVSQNYPIVQNIQ

SEQ ID NO: 33 - protein, 15 aa, Human Immunodeficiency Virus
SQNYPIVQNIQGQMV

SEQ ID NO: 34 - protein, 15 aa, Human Immunodeficiency Virus
PIVQNIQGQMVHQAI

SEQ ID NO: 35 - protein, 15 aa, Human Immunodeficiency Virus
NIQGQMVHQAISPRT

SEQ ID NO: 36 - protein, 15 aa, Human Immunodeficiency Virus
QMVHQAISPRTLNAW

SEQ ID NO: 37 - protein, 15 aa, Human Immunodeficiency Virus
QAISPRTLNAWVKVV

SEQ ID NO: 38 - protein, 15 aa, Human Immunodeficiency Virus
PRTLNAWVKVVEEKA

SEQ ID NO: 39 - protein, 15 aa, Human Immunodeficiency Virus
NAWVKVVEEKAFSPE

SEQ ID NO: 40 - protein, 15 aa, Human Immunodeficiency Virus
KVVEEKAFSPEVIPM

SEQ ID NO: 41 - protein, 15 aa, Human Immunodeficiency Virus
EKAFSPEVIPMFSAL

SEQ ID NO: 42 - protein, 15 aa, Human Immunodeficiency Virus
SPEVIPMFSALSEGA

SEQ ID NO: 43 - protein, 15 aa, Human Immunodeficiency Virus
IPMFSALSEGATPQD

SEQ ID NO: 44 - protein, 15 aa, Human Immunodeficiency Virus
SALSEGATPQDLNTM

SEQ ID NO: 45 - protein, 15 aa, Human Immunodeficiency Virus
EGATPQDLNTMLNTV

SEQ ID NO: 46 - protein, 15 aa, Human Immunodeficiency Virus
PQDLNTMLNTVGGHQ

SEQ ID NO: 47 - protein, 15 aa, Human Immunodeficiency Virus
NTMLNTVGGHQAAMQ

SEQ ID NO: 48 - protein, 15 aa, Human Immunodeficiency Virus
NTVGGHQAAMQMLKE

SEQ ID NO: 49 - protein, 15 aa, Human Immunodeficiency Virus
GHQAAMQMLKETINE

SEQ ID NO: 50 - protein, 15 aa, Human Immunodeficiency Virus
AMQMLKETINEEAAE

SEQ ID NO: 51 - protein, 15 aa, Human Immunodeficiency Virus
LKETINEEAAEWDRV

SEQ ID NO: 52 - protein, 15 aa, Human Immunodeficiency Virus
INEEAAEWDRVHPVH

SEQ ID NO: 53 - protein, 15 aa, Human Immunodeficiency Virus
AAEWDRVHPVHAGPI

SEQ ID NO: 54 - protein, 15 aa, Human Immunodeficiency Virus
DRVHPVHAGPIAPGQ

SEQ ID NO: 55 - protein, 15 aa, Human Immunodeficiency Virus
PVHAGPIAPGQMREP

SEQ ID NO: 56 - protein, 15 aa, Human Immunodeficiency Virus
GPIAPGQMREPRGSD

SEQ ID NO: 57 - protein, 15 aa, Human Immunodeficiency Virus
PGQMREPRGSDIAGT

SEQ ID NO: 58 - protein, 15 aa, Human Immunodeficiency Virus
REPRGSDIAGTTSTL

SEQ ID NO: 59 - protein, 15 aa, Human Immunodeficiency Virus
GSDIAGTTSTLQEQI

SEQ ID NO: 60 - protein, 15 aa, Human Immunodeficiency Virus
AGTTSTLQEQIGWMT

SEQ ID NO: 61 - protein, 15 aa, Human Immunodeficiency Virus
STLQEQIGWMTNNPP

SEQ ID NO: 62 - protein, 15 aa, Human Immunodeficiency Virus
EQIGWMTNNPPIPVG

SEQ ID NO: 63 - protein, 15 aa, Human Immunodeficiency Virus
WMTNNPPIPVGEIYK

SEQ ID NO: 64 - protein, 15 aa, Human Immunodeficiency Virus
NPPIPVGEIYKRWII

SEQ ID NO: 65 - protein, 15 aa, Human Immunodeficiency Virus
PVGEIYKRWIILGLN

SEQ ID NO: 66 - protein, 15 aa, Human Immunodeficiency Virus
IYKRWIILGLNKIVR

SEQ ID NO: 67 - protein, 15 aa, Human Immunodeficiency Virus
WIILGLNKIVRMYSP

SEQ ID NO: 68 - protein, 15 aa, Human Immunodeficiency Virus
GLNKIVRMYSPTSIL

SEQ ID NO: 69 - protein, 15 aa, Human Immunodeficiency Virus
IVRMYSPTSILDIRQ

SEQ ID NO: 70 - protein, 15 aa, Human Immunodeficiency Virus
YSPTSILDIRQGPKE

SEQ ID NO: 71 - protein, 15 aa, Human Immunodeficiency Virus
SILDIRQGPKEPFRD

SEQ ID NO: 72 - protein, 15 aa, Human Immunodeficiency Virus
IRQGPKEPFRDYVDR

SEQ ID NO: 73 - protein, 15 aa, Human Immunodeficiency Virus
PKEPFRDYVDRFYKT

SEQ ID NO: 74 - protein, 15 aa, Human Immunodeficiency Virus
FRDYVDRFYKTLRAE

SEQ ID NO: 75 - protein, 15 aa, Human Immunodeficiency Virus
VDRFYKTLRAEQASQ

SEQ ID NO: 76 - protein, 15 aa, Human Immunodeficiency Virus
YKTLRAEQASQEVKN

SEQ ID NO: 77 - protein, 15 aa, Human Immunodeficiency Virus
RAEQASQEVKNWMTE

SEQ ID NO: 78 - protein, 15 aa, Human Immunodeficiency Virus
ASQEVKNWMTETLLV

SEQ ID NO: 79 - protein, 15 aa, Human Immunodeficiency Virus
VKNWMTETLLVQNAN

SEQ ID NO: 80 - protein, 15 aa, Human Immunodeficiency Virus
MTETLLVQNANPDCK

SEQ ID NO: 81 - protein, 15 aa, Human Immunodeficiency Virus
LLVQNANPDCKTILK

SEQ ID NO: 82 - protein, 15 aa, Human Immunodeficiency Virus
NANPDCKTILKALGP

SEQ ID NO: 83 - protein, 15 aa, Human Immunodeficiency Virus
DCKTILKALGPAATL

SEQ ID NO: 84 - protein, 15 aa, Human Immunodeficiency Virus
ILKALGPAATLEEMM

SEQ ID NO: 85 - protein, 15 aa, Human Immunodeficiency Virus
LGPAATLEEMMTACQ

SEQ ID NO: 86 - protein, 15 aa, Human Immunodeficiency Virus
ATLEEMMTACQGVGG

SEQ ID NO: 87 - protein, 15 aa, Human Immunodeficiency Virus
EMMTACQGVGGPGHK

SEQ ID NO: 88 - protein, 15 aa, Human Immunodeficiency Virus
ACQGVGGPGHKARVL

SEQ ID NO: 89 - protein, 15 aa, Human Immunodeficiency Virus
VGGPGHKARVLAEAM

SEQ ID NO: 90 - protein, 15 aa, Human Immunodeficiency Virus
GHKARVLAEAMSQVT

SEQ ID NO: 91 - protein, 15 aa, Human Immunodeficiency Virus
RVLAEAMSQVTNSAT

SEQ ID NO: 92 - protein, 15 aa, Human Immunodeficiency Virus
EAMSQVTNSATIMMQ

SEQ ID NO: 93 - protein, 15 aa, Human Immunodeficiency Virus
QVTNSATIMMQRGNF

SEQ ID NO: 94 - protein, 15 aa, Human Immunodeficiency Virus
SATIMMQRGNFRNQR

SEQ ID NO: 95 - protein, 15 aa, Human Immunodeficiency Virus
MMQRGNFRNQRKIVK

SEQ ID NO: 96 - protein, 15 aa, Human Immunodeficiency Virus
GNFRNQRKIVKCFNC

SEQ ID NO: 97 - protein, 15 aa, Human Immunodeficiency Virus
NQRKIVKCFNCGKEG

SEQ ID NO: 98 - protein, 15 aa, Human Immunodeficiency Virus
IVKCFNCGKEGHTAR

SEQ ID NO: 99 - protein, 15 aa, Human Immunodeficiency Virus
FNCGKEGHTARNCRA

SEQ ID NO: 100 - protein, 15 aa, Human Immunodeficiency Virus
KEGHTARNCRAPRKK

SEQ ID NO: 101 - protein, 15 aa, Human Immunodeficiency Virus
TARNCRAPRKKGCWK

SEQ ID NO: 102 - protein, 15 aa, Human Immunodeficiency Virus
CRAPRKKGCWKCGKE

SEQ ID NO: 103 - protein, 15 aa, Human Immunodeficiency Virus
RKKGCWKCGKEGHQM

SEQ ID NO: 104 - protein, 15 aa, Human Immunodeficiency Virus
CWKCGKEGHQMKDCT

SEQ ID NO: 105 - protein, 15 aa, Human Immunodeficiency Virus
GKEGHQMKDCTERQA

SEQ ID NO: 106 - protein, 15 aa, Human Immunodeficiency Virus
HQMKDCTERQANFLG

SEQ ID NO: 107 - protein, 15 aa, Human Immunodeficiency Virus
DCTERQANFLGKIWP

SEQ ID NO: 108 - protein, 15 aa, Human Immunodeficiency Virus
RQANFLGKIWPSYKG

SEQ ID NO: 109 - protein, 15 aa, Human Immunodeficiency Virus
FLGKIWPSYKGRPGN

SEQ ID NO: 110 - protein, 15 aa, Human Immunodeficiency Virus
IWPSYKGRPGNFLQS

SEQ ID NO: 111 - protein, 15 aa, Human Immunodeficiency Virus
YKGRPGNFLQSRPEP

SEQ ID NO: 112 - protein, 15 aa, Human Immunodeficiency Virus
PGNFLQSRPEPTAPP

SEQ ID NO: 113 - protein, 15 aa, Human Immunodeficiency Virus
LQSRPEPTAPPEESF

SEQ ID NO: 114 - protein, 15 aa, Human Immunodeficiency Virus
PEPTAPPEESFRSGV

SEQ ID NO: 115 - protein, 15 aa, Human Immunodeficiency Virus
APPEESFRSGVETTT

SEQ ID NO: 116 - protein, 15 aa, Human Immunodeficiency Virus
ESFRSGVETTTPPQK

SEQ ID NO: 117 - protein, 15 aa, Human Immunodeficiency Virus
SGVETTTPPQKQEPI

SEQ ID NO: 118 - protein, 15 aa, Human Immunodeficiency Virus
TTTPPQKQEPIDKEL

SEQ ID NO: 119 - protein, 15 aa, Human Immunodeficiency Virus
PQKQEPIDKELYPLT

SEQ ID NO: 120 - protein, 15 aa, Human Immunodeficiency Virus
EPIDKELYPLTSLRS

SEQ ID NO: 121 - protein, 15 aa, Human Immunodeficiency Virus
KELYPLTSLRSLFGN

SEQ ID NO: 122 - protein, 16 aa, Human Immunodeficiency Virus
PLTSLRSLFGNDPSSQ

SEQ ID NO: 123 - protein, 20 aa, Simian Immunodeficiency Virus
MGCLGNQLLIAILLLSVYGI

SEQ ID NO: 124 - protein, 20 aa, Simian Immunodeficiency Virus
AILLLSVYGIYCTLYVTVFY

SEQ ID NO: 125 - protein, 20 aa, Simian Immunodeficiency Virus
YCTLYVTVFYGVPAWRNATI

SEQ ID NO: 126 - protein, 20 aa, Simian Immunodeficiency Virus
GVPAWRNATIPLFCATKNRD

SEQ ID NO: 127 - protein, 20 aa, Simian Immunodeficiency Virus
PLFCATKNRDTWGTTQCLPD

SEQ ID NO: 128 - protein, 20 aa, Simian Immunodeficiency Virus
TWGTTQCLPDNGDYSEVALN

SEQ ID NO: 129 - protein, 20 aa, Simian Immunodeficiency Virus
NGDYSEVALNVTESFDAWNN

SEQ ID NO: 130 - protein, 20 aa, Simian Immunodeficiency Virus
VTESFDAWNNTVTEQAIEDV

SEQ ID NO: 131 - protein, 20 aa, Simian Immunodeficiency Virus
TVTEQAIEDVWQLFETSIKP

SEQ ID NO: 132 - protein, 20 aa, Simian Immunodeficiency Virus
WQLFETSIKPCVKLSPLCIT

SEQ ID NO: 133 - protein, 20 aa, Simian Immunodeficiency Virus
CVKLSPLCITMRCNKSETDR

SEQ ID NO: 134 - protein, 20 aa, Simian Immunodeficiency Virus
MRCNKSETDRWGLTKSITTT

SEQ ID NO: 135 - protein, 20 aa, Simian Immunodeficiency Virus
WGLTKSITTTASTTSTTASA

SEQ ID NO: 136 - protein, 20 aa, Simian Immunodeficiency Virus
ASTTSTTASAKVDMVNETSS

SEQ ID NO: 137 - protein, 20 aa, Simian Immunodeficiency Virus
KVDMVNETSSCIAQDNCTGL

SEQ ID NO: 138 - protein, 20 aa, Simian Immunodeficiency Virus
CIAQDNCTGLEQEQMISCKF

SEQ ID NO: 139 - protein, 20 aa, Simian Immunodeficiency Virus
EQEQMISCKFNMTGLKRDKK

SEQ ID NO: 140 - protein, 20 aa, Simian Immunodeficiency Virus
NMTGLKRDKKKEYNETWYSA

SEQ ID NO: 141 - protein, 20 aa, Simian Immunodeficiency Virus
KEYNETWYSADLVCEQGNNT

SEQ ID NO: 142 - protein, 20 aa, Simian Immunodeficiency Virus
DLVCEQGNNTGNESRCYMNH

SEQ ID NO: 143 - protein, 20 aa, Simian Immunodeficiency Virus
GNESRCYMNHCNTSVIQESC

SEQ ID NO: 144 - protein, 20 aa, Simian Immunodeficiency Virus
CNTSVIQESCDKHYWDAIRF

SEQ ID NO: 145 - protein, 20 aa, Simian Immunodeficiency Virus
DKHYWDAIRFRYCAPPGYAL

SEQ ID NO: 146 - protein, 20 aa, Simian Immunodeficiency Virus
RYCAPPGYALLRCNDTNYSG

SEQ ID NO: 147 - protein, 20 aa, Simian Immunodeficiency Virus
LRCNDTNYSGFMPKCSKVVV

SEQ ID NO: 148 - protein, 20 aa, Simian Immunodeficiency Virus
FMPKCSKVVVSSCTRMMETQ

SEQ ID NO: 149 - protein, 20 aa, Simian Immunodeficiency Virus
SSCTRMMETQTSTWFGFNGT

SEQ ID NO: 150 - protein, 20 aa, Simian Immunodeficiency Virus
TSTWFGFNGTRAENRTYIYW

SEQ ID NO: 151 - protein, 20 aa, Simian Immunodeficiency Virus
RAENRTYIYWHGRDNRTIIS

SEQ ID NO: 152 - protein, 20 aa, Simian Immunodeficiency Virus
HGRDNRTIISLNKYYNLTMK

SEQ ID NO: 153 - protein, 20 aa, Simian Immunodeficiency Virus
LNKYYNLTMKCRRPGNKTVL

SEQ ID NO: 154 - protein, 20 aa, Simian Immunodeficiency Virus
CRRPGNKTVLPVTIMSGLVF

SEQ ID NO: 155 - protein, 20 aa, Simian Immunodeficiency Virus
PVTIMSGLVFHSQPINDRPK

SEQ ID NO: 156 - protein, 20 aa, Simian Immunodeficiency Virus
HSQPINDRPKQAWCWFGGKW

SEQ ID NO: 157 - protein, 20 aa, Simian Immunodeficiency Virus
QAWCWFGGKWKDAIKEVKQT

SEQ ID NO: 158 - protein, 20 aa, Simian Immunodeficiency Virus
KDAIKEVKQTIVKHPRYTGT

SEQ ID NO: 159 - protein, 20 aa, Simian Immunodeficiency Virus
IVKHPRYTGTNNTDKINLTA

SEQ ID NO: 160 - protein, 20 aa, Simian Immunodeficiency Virus
NNTDKINLTAPGGGDPEVTF

SEQ ID NO: 161 - protein, 20 aa, Simian Immunodeficiency Virus
PGGGDPEVTFMWTNCRGEFL

SEQ ID NO: 162 - protein, 20 aa, Simian Immunodeficiency Virus
MWTNCRGEFLYCKMNWFLNW

SEQ ID NO: 163 - protein, 20 aa, Simian Immunodeficiency Virus
YCKMNWFLNWVEDRNTANQK

SEQ ID NO: 164 - protein, 20 aa, Simian Immunodeficiency Virus
VEDRNTANQKPKEQHKRNYV

SEQ ID NO: 165 - protein, 20 aa, Simian Immunodeficiency Virus
PKEQHKRNYVPCHIRQIINT

SEQ ID NO: 166 - protein, 20 aa, Simian Immunodeficiency Virus
PCHIRQIINTWHKVGKNVYL

SEQ ID NO: 167 - protein, 20 aa, Simian Immunodeficiency Virus
WHKVGKNVYLPPREGDLTCN

SEQ ID NO: 168 - protein, 20 aa, Simian Immunodeficiency Virus
PPREGDLTCNSTVTSLIANI

SEQ ID NO: 169 - protein, 20 aa, Simian Immunodeficiency Virus
STVTSLIANIDWIDGNQTNI

SEQ ID NO: 170 - protein, 20 aa, Simian Immunodeficiency Virus
DWIDGNQTNITMSAEVAELY

SEQ ID NO: 171 - protein, 20 aa, Simian Immunodeficiency Virus
TMSAEVAELYRLELGDYKLV

SEQ ID NO: 172 - protein, 20 aa, Simian Immunodeficiency Virus
RLELGDYKLVEITPIGLAPT

SEQ ID NO: 173 - protein, 20 aa, Simian Immunodeficiency Virus
EITPIGLAPTDVKRYTTGGT

SEQ ID NO: 174 - protein, 20 aa, Simian Immunodeficiency Virus
DVKRYTTGGTSRNKRGVFVL

SEQ ID NO: 175 - protein, 20 aa, Simian Immunodeficiency Virus
SRNKRGVFVLGFLGFLATAG

SEQ ID NO: 176 - protein, 20 aa, Simian Immunodeficiency Virus
GFLGFLATAGSAMGAASLTL

SEQ ID NO: 177 - protein, 20 aa, Simian Immunodeficiency Virus
SAMGAASLTLTAQSRTLLAG

SEQ ID NO: 178 - protein, 20 aa, Simian Immunodeficiency Virus
TAQSRTLLAGIVQQQQQLLD

SEQ ID NO: 179 - protein, 20 aa, Simian Immunodeficiency Virus
IVQQQQQLLDVVKRQQELLR

SEQ ID NO: 180 - protein, 20 aa, Simian Immunodeficiency Virus
VVKRQQELLRLTVWGTKNLQ

SEQ ID NO: 181 - protein, 20 aa, Simian Immunodeficiency Virus
LTVWGTKNLQTRVTAIEKYL

SEQ ID NO: 182 - protein, 20 aa, Simian Immunodeficiency Virus
TRVTAIEKYLKDQAQLNAWG

SEQ ID NO: 183 - protein, 20 aa, Simian Immunodeficiency Virus
KDQAQLNAWGCAFRQVCHTT

SEQ ID NO: 184 - protein, 20 aa, Simian Immunodeficiency Virus
CAFRQVCHTTVPWPNASLTP

SEQ ID NO: 185 - protein, 20 aa, Simian Immunodeficiency Virus
VPWPNASLTPKWNNETWQEW

SEQ ID NO: 186 - protein, 20 aa, Simian Immunodeficiency Virus
KWNNETWQEWERKVDFLEEN

SEQ ID NO: 187 - protein, 20 aa, Simian Immunodeficiency Virus
ERKVDFLEENITALLEEAQI

SEQ ID NO: 188 - protein, 20 aa, Simian Immunodeficiency Virus
ITALLEEAQIQQEKNMYELQ

SEQ ID NO: 189 - protein, 20 aa, Simian Immunodeficiency Virus
QQEKNMYELQKLNSWDVFGN

SEQ ID NO: 190 - protein, 20 aa, Simian Immunodeficiency Virus
KLNSWDVFGNWFDLASWIKY

SEQ ID NO: 191 - protein, 20 aa, Simian Immunodeficiency Virus
WFDLASWIKYIQYGVYIVVG

SEQ ID NO: 192 - protein, 20 aa, Simian Immunodeficiency Virus
IQYGVYIVVGVILLRIVIYI

SEQ ID NO: 193 - protein, 20 aa, Simian Immunodeficiency Virus
VILLRIVIYIVQMLAKLRQG

SEQ ID NO: 194 - protein, 20 aa, Simian Immunodeficiency Virus
VQMLAKLRQGYRPVFSSPPS

SEQ ID NO: 195 - protein, 20 aa, Simian Immunodeficiency Virus
YRPVFSSPPSYFQQTHIQQD

SEQ ID NO: 196 - protein, 20 aa, Simian Immunodeficiency Virus
YFQQTHIQQDPALPTREGKE

SEQ ID NO: 197 - protein, 20 aa, Simian Immunodeficiency Virus
PALPTREGKERDGGEGGGNS

SEQ ID NO: 198 - protein, 20 aa, Simian Immunodeficiency Virus
RDGGEGGGNSSWPWQIEYIH

SEQ ID NO: 199 - protein, 20 aa, Simian Immunodeficiency Virus
SWPWQIEYIHFLIRQLIRLL

SEQ ID NO: 200 - protein, 20 aa, Simian Immunodeficiency Virus
FLIRQLIRLLTWLFSNCRTL

SEQ ID NO: 201 - protein, 20 aa, Simian Immunodeficiency Virus
TWLFSNCRTLLSRVYQILQP

SEQ ID NO: 202 - protein, 20 aa, Simian Immunodeficiency Virus
LSRVYQILQPILQRLSATLQ

SEQ ID NO: 203 - protein, 20 aa, Simian Immunodeficiency Virus
ILQRLSATLQRIREVLRTEL

SEQ ID NO: 204 - protein, 20 aa, Simian Immunodeficiency Virus
RIREVLRTELTYLQYGWSYF

SEQ ID NO: 205 - protein, 20 aa, Simian Immunodeficiency Virus
TYLQYGWSYFHEAVQAVWRS

SEQ ID NO: 206 - protein, 20 aa, Simian Immunodeficiency Virus
HEAVQAVWRSATETLAGAWG

SEQ ID NO: 207 - protein, 9 aa, Human Immunodeficiency Virus
AMQMLKETI

SEQ ID NO: 208 - protein, 9 aa, Human Immunodeficiency Virus
GPGQAFYAT

SEQ ID NO: 209 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 210 - protein, 588 aa, Ebola virus
  VARIANT 120: Xaa = Any Amino Acid
HGFRFEVKKRDGVKRLEELLPAVSSGKNIKRTLAAMPEEETTEANAGQFLSFASLFLPKLVVGEKACLEKVQRQIQVHAEQGLIQYPTAWQSVGHMMVIFRLMRTNFLIKFLLIHQGMHXVAGHDANDAVISNSVAQARFSGLLIVKTVLDHILQKTERGVRLHPLARTAKVKNEVNSFKAALSSLAKHGEYAPFARLLNLSGVNNLEHGLFPQLSAIALGVATAHGSTLAGVNVGEQYQQLREAATEAEKQLQQYAESRELDHLGLDDQEKKILMNFHQKKNEISFQQTNAMVTLRKERLAKLTEAITAASLPKTSGHYDDDDDIPFPGPINDDDNPGHQDDDPTDSQDTTIPDVVVDPDDGSYGEYQSYSENGMNAPDDLVLFDLDEDDEDTKPVPNRSTKGGQQKNSQKGQHTEGRQTQSRPTQNVPGPHRTIHHASAPLTDNDRRNEPSGSTSPRMLTPINEEADPLDDADDETSSLPPLESDDEEQDRGGTSNRTPTVAPPAPVYRDHSEKKELPQDERQDQDHTQEARNQDSDNTQPEHSFEEMYRHILRSQGPFDAVLYYHMMKDEPVVFSTSDGKEYTYP

SEQ ID NO: 211 - protein, 2280 aa, Hepatitis C Virus
MSTNPKPQRKTKRNTYRRPQDVKFPGGGQIVGGVYVLPRRGPTLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYQVRNASGLYHVTNDCSNSSIVYEAAGMIMHTPGCVPCVRENNASRCWVALTPTLAARNTSIPTTTIRRHVDLLVGAAAFCSAMYVGDLCGSVFLVSQLFTFSPRRYETVQDCNCSIYPGHVSGHRMAWDMMMNWSPTTALVVSQLLRIPQAVVDMVAGAHWGVLAGLAYYSMVGNWAKVLIVMLLFAGVDGVTYTTGGSQARHTQSVTSFFTQGPAQRIQLINTNGSWHINRTALNCNESLNTGFFAALFYAHKFNSSGCPERMASCSSIDKFAQGWGPITYTEPRDLDQRPYCWHYAPRQCGIVPASQVCGPVYCFTPSPVVVGTTDRSGAPTYNWGANETDVLLLNNTRPPQGNWFGCTWMNSTGFTKTCGGPPCNIGGVGNLTLTCPTDCFRKHPEATYTKCGSGPWLTPRCIVDYPYRLWHYPCTVNFTIFKVRMYVGGVEHRLSAACNWTRGERCDLEDRDRSELSPLLLSTTEWQTLPCSFTTLPALSTGLIHLHQNIVDVQYLYGIGSAVVSFVIKWEYIVLLFLLLADARVCACLWMMLLIAQAEAALENLVVLNAASLAGADGILSFLVFFCAAWYIKGRLVPGAAYALYGVWPLLLLLLALPPRAYAMDREMAASCGGVVFVGLILLTLSPHYKVFLARLIWWLQYFITRAEAHLCVWVPPLNVRGGRDAIILLTCAAHPELIFDITKLLLAILGPLMVLQAAITAMPYFVRAQGLIRACMLVRKVAGGHYVQMAFMKLAALTGTYVYDHLTPLQDWAHAGLRDLAVAVEPVVFSDMETKIITWGADTAACGDIILGLPVSARRGREILLGPADSIEGQGWRLLAPITAYAQQTRGLLGCIVTSLTGRDKNQVEGEVQVVSTATQSFLATCVNGVCWTVFHGAGSKTLAGPKGPITQMYTNVDQDLVGWHAPPGARSLTPCTCGSSDLYLVTRHADVIPVRRRGDGRGSLLSPRPVSYLKGSSGGPLLCPSGHAVGIFRAAVCTRGVAKAVDFIPVESMETTMRSPVFTDNSSPPAVPQTFQVAHLHAPTGSGKSTKVPAAYAAQGYKVLVLNPSVAATLGFGAYMSKAHGTDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPLEAIKGGRHLIFCHSKKKCDELAAKLSGLGINAVAYYRGLDVSVIPTSGDVVIVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETTVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEITLTHPITKFIMACMSADLEVVTSTWVLVGGVLAALAAYCLTTGSVVIVGRIILSGRPAVVPDREVLYREFDEMEECASHLPYIEQGMQLAEQFKQKALGLLQTATKQAEAAAPVVESRWRALEAFWAKHMWNFISGIQYLAGLSTLPGNPAIASLMAFTASITSPLTTQNTLLFNILGGWVAAQLAPPSAASAFVGAGIAGAAIGSIGLGKVLVDILAGYGAGVAGALVAFKVMSGEAPSAEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLKDVWDWICTVLTDFKTWLQSKLLPKLPGVPFFSCQRGYKGVWRGDGIMQTTCPCGAQITGHVKNGSMRIVGPKTCSNTWHGTFPINAYTTGPCTPSPAPNYSRALWRVAAEEYVEITRVGDFHYVTGMTTDNVKCPCQVPAPEFFTELDGVRLHRYAPACRPLLREDVTFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESENKVVILDSFDPLRAEEDEREVSVAAEILRKSKK

SEQ ID NO: 212 - protein, 128 aa, Homo sapiens
MRTLDLIDEAYGLDFYILKTPKEDLCSKFGMELKRGMLLRLARQDPQLHPEDPERRAAIYDKYKEFAIPEEEAEWVGLTLEEAIEKQRLLEEKDPVPLFKIYVAELIQQLQQQALSEPAVVQKTASGQ

SEQ ID NO: 213 - protein, 1255 aa, Homo sapiens [sequence omitted]

SEQ ID NO: 214 - protein, 574 aa, Respiratory Syncytial Virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 215 - protein, 151 aa, Human Immunodeficiency Virus 2
DVVKRQQELLRLTVWGTKNLQARVTAIEKYLKDQAHVNSWGCAFRQVCHTTVPWVNDTLTPDWDNMTWQEWEEKVRYLEANISQSLEQAQILQEKNMYELQKLNSWDIFGNWFDLTSWVKYIQYGVCIIVGIVALRIVIYVVQMLSRLRKG

SEQ ID NO: 216 - protein, 522 aa, Human Immunodeficiency Virus
MGARNSVLRGKKADELERIRLRPGGKKKYRLKHIVWAANKLDRFGLAESLLESKEGCQKILTVLDPMVPTGSENLKSLFNTVCVIWCIHAEEKVKDTEGAKQIVRRHLVAETGTAEKMPSTSRPTAPSSEKGGNYPVQHVGGNYTHIPLSPRTLNAWVKLVEEKKFGAEVVPGFQALSEGCTPYDINQMLNCVGDHQAAMQIIREIINEEAAEWDVQHPIPGPLPAGQLREPRGSDIAGTTSTVEEQIQWMFRPQNPVPVGNIYRRWIQIGLQKCVRMYNPTNILDIKQGPKEPFQSYVDRFYKSLRAEQTDPAVKNWMTQTLLVQNANPDCKLVLKGLGMNPTLEEMLTACQGVGGPGQKARLMAEALKEVIGPAPIPFAAAQQRKAFKCWNCGKEGHSARQCRAPRRQGCWKCGKPGHIMTNCPDRQAGFLGLGPWGKKPRNFPVAQVPQGLTPTAPPVDPAVDLLEKYMQQGKRQREQRERPYKEVTEDLLHLEQGETPYREPPTEDLLHLNSLFGKDQ

SEQ ID NO: 217 - protein, 860 aa, Human Immunodeficiency Virus 2
MEPGRNQLFVVILLTSACLVYCSQYVTVFYGIPAWKNASIPLFCATKNRDTWGTIQCLPDNDDYQEIILNVTEAFDAWNNTVTEQAVEDVWHLFETSIKPCVKLTPLCVAMNCSRVQGNTTTPNPRTSSSTTSRPPTSAASIINETSNCIENNTCAGLGYEEMMQCEFNMKGLEQDKKRRYKDTWYLEDVVCDNTTAGTCYMRHCNTSIIKESCDKHYWDAMRFRYCAPPGFALLRCNDTNYSGFEPKCTKVVAASCTRMMETQTSTWFGFNGTRAENRTYIYWHGRDNRTIISLNKYYNLTMRCKRPGNKTVLPITLMSGLVFHSQPINTRPRQAWCRFGGRWREAMQEVKQTLVQHPRYKGINDTGKINFTKPGAGSDPEVAFMWTNCRGEFLYCNMTWFLNWVEDKNQTRRNYCHIKQIINTWHKVGKNVYLPPREGELACESTVTSIIANIDIDKNRTHTNITFSAEVAELYRLELGDYKLIEITPIGFAPTDQRRYSSTPVRNKRGVFVLGFLGFLATAGSAMGARSLTLSAQSRTLLAGIVQQQQQLLDVVKRQQEMLRLTVWGTKNLQARVTAIEKYLKHQAQLNSWGCAFRQVCHTTVPWVNDSLSPDWKNMTWQEWEKQVRYLEANISQSLEEAQIQQEKNMYELQKLNSWDILGNWFDLTSWVKYIQYGVHIVVGIIALRIAIYVVQLLSRFRKGYRPVFSSPPGYLQQIHIHKDRGQPANEGTEEDVGGDSGYDLWPWPINYVQFLIHLLTRLLIGLYNICRDLLSKNSPTRRLISQSLTAIRDWLRLKAAQLQYGCEWIQEAFQAFARTTRETLAGAWGWLWEAARRIGRGILAVPRRIRQGAELALL

SEQ ID NO: 218 - protein, 25 aa, Human Immunodeficiency Virus
SEGDTDELAKLVEMGNYDLGDASDL

SEQ ID NO: 219 - protein, 854 aa, Human Immunodeficiency Virus
MRVKGIMRNCQQWWIWGILGFWMLLICNGEGNLWVTVYYGVPVWKEAKTTLFCASDAKGYEREVHNIWATHACVPTDPNPQEMFLHNVTENFNMWKNDMVDQMHEDIISLWDESLKPCVKLTPLCVTLECKNVTTNVTINNATSVTANNNTSDMKNCSFNATTEVTDKIRKENALFYTLDIVPLDENQNNSNYRLINCNTSKVTQACPKVSFDPIPLHYCAPAGYAILKCNNNTFNGTGPCNNVSTIQCTHGIKPVVSTQLLLNGSRAEKEIIIRSENMTNNAKTIIVHLNESIEIECIRPNNNTRKSIRIGPGQTFYATNGMIGDIRQAHCNISGADWNRTLQGVGRKLAGYFPNKTISFQPSSGGDLEITTHSFNCGGEFFYCNTSSLFNNTYRPTYWPNGTESNSTITLQCRIKQIINMWQKVGRAIYAPPIAGKITCKSNITGLLLVRDGGNGGNNTATEIFRPGGGNMKDNWRSELYKYKVVEIKPLGIAPTGAKRRVVGREKRAVGIGAVFLGFLGAAGSTMGAASITLTVQARQLLSGIVQQQSNLLKAIEAQHHLLQLTVWGIKQLQARVLAIERYLKDQQLLGIWGCSGKLICTTAVPWNSSWSNKSQADIWDNMTWMQWDREISNYTDTIYRLLEVSQTQQEQNEQDLLALNKWQHLWNWFDITKWLWYIKIFIMIVGGLIGLRIIFAVLSIVNRVRQGYSPLSLQTLTPNQREPDRLGRIEEEGGEQDRKRSIRLVSGFLALAWDDLRSLCLFSYHHLRDFILIAARVVELLGRRGWDILKYLASLVQYWGLELKKGAISLLDSIAIAVAEGTDRIIAFIQRLFRAICNLPRRIRQGFEASLL

SEQ ID NO: 220 - protein, 242 aa, Human Immunodeficiency Virus
MAETEALSKLREDFRMQNKSVFILGASGETGRVLLKEILEQGLFSKVTLIGRRKLTFDEEAYKNVNQEVVDFEKLDDYASAFQGHDVGFCCLGTTRGKAGAEGFVRVDRDYVLKSAELAKAGGCKHFNLLSSKGADKSSNFLYLQVKGEVEAKVEELKFDRYSVFRPGVLLCDRQESRPGEWLVRKFFGSLPDSWARGHSVPVVTVVRAMLNNVVRPRDKQMELLENKAIHDLGKAHGSLKP

SEQ ID NO: 221 - protein, 210 aa, Human Immunodeficiency Virus
  VARIANT 31, 97, 140, 141, 144, 178: Xaa = Any Amino Acid
CTEMEKEGKISKIGPENPYNTPVFAIKKKDXTKWRKLVDFRELNKRTQDFWEVQLGIPHPAGLKKKKSVTVLDVGDAYFSVPLDESFRKYTAFTIPXTNNETPGIRYQYNVLPQGWKGSPAIFQSSMTKILEPFRIKNPXXVIXQYMDDLYVGSDLEIGQHRAKIEELRKHLLSWGFXTPDKKHQKEPPFLWMGYELHPDKWTVQPIQLP

SEQ ID NO: 222 - protein, 207 aa, Human Immunodeficiency Virus
MGGKWSKSSLVGWPEVRDRIRRTDPAAEGVGAASQDLDKHGALTNSNTAATNKDCAWLEAQEEEGEVGFPVRPQVPLRPMTYKGAFDLGWFLKEKGGLDGLIYSKKRQEILDLWVYHTQGYFPDWQNYTPGPGVRYPLTFGWCYKLVPVDPKEVEEATEGENNCLLHPICQHGMEDEDREVLRWKFDSELARRHIARERHPEFYKDC

SEQ ID NO: 223 - protein, 397 aa, Plasmodium falciparum
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 224 - protein, 69 aa, Plasmodium falciparum
TEWSPCSVTCGNGIQVRIKPGSANKPKDELDYENDIEKKICKMEKCSSVFNVVNSSIGLIMVLSFLFLN

SEQ ID NO: 225 - protein, 137 aa, Salmonella enterica
MYMSKYVPVYTLLILIYSFNASAEWTGDNTNAYYSDEVISELHVGQIDTSPYFCIKTVKANGSGTPVVACAVSKQSIWAPSFKELLDQARYFYSTGQSVRIHVQKNIWTYPLFVNTFSANALVGLSSCSATQCFGPK

SEQ ID NO: 226 - protein, 233 aa, Staphylococcus aureus
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKSINSENMHIDIYLYTS

SEQ ID NO: 227 - protein, 68 aa, Escherichia coli
AWREEPWIHHAPQGCGDSSRTITGDTCNEETQNLSTIYLRKYQSKVKRQIFSDYQSEVDIYNRIRNEL

SEQ ID NO: 228 - protein, 396 aa, Clostridium difficile
NEYYPEIIVLNPNTFHKKVNINLDSSSFEYKWSTEGSDFILVRYLEESNKKILQKIRIKGILSNTKSFNKMSIDFKDIKKLSLGYIMSNFKSFNSENELDRDHLGFKIIDNKTYYYDEASKLVKGLININNSLFYFDPIESNLVTGWQTINGKKYYFDINTGAASTSYKIINGKHFYFNNNGVMQLGVFKGPDGFEYFAPANTQNNNIEGQAIVYQSKFLTLNGKKYYFDNDSKAVTGWQTIDGKKYYFNLNTAEAATGWQTIDGKKYYFNTNTSIASTGYTIINGKHFYFNTDGIMQIGVFKGPNGFEYFAPANTDANNIEGQAIRYQNRFLYLHDNIYYFGNNSKAVTGWQTINGNVYYFMPDTAMAAAGGLFEIDGVIYFFGVDGVKAPGIYG

SEQ ID NO: 229 - protein, 386 aa, Bacillus cereus
MKKTLITGLLVTAVSTSRFIPVSAYAKEGQTEVKTVYAQNVIAPNTLSNSIRMLGSQSPLIQAYGLIILQQPDIKVNAMSSLTNHQKFAKANVREWIDEYNPKLIDLNQEMMRYSTRFNSYYSKLYELAGNVNEDQQAKADFMSAYGKLQLQVQSIQESMEQDLLELNRFKTVLDKDSNNLSIKADEAIKTLQGSSGDIVKLREDIKRIQGEIQAELTTILNRPQEIIKGSINIGKQVFTITNQTAQTKTIDFVSIGTLSNEIVNAADSQTREAALRIQQKQKELLPLIQKLSQTEAEATQITFVEDQVNSFTELIDRQITTLETLLTDWKVLNNNMIQIQKNVEEGTYTDSSLLQKHFNQIKKVSDEMNKQTNQFEDYVTNVEVH

SEQ ID NO: 230 - protein, 227 aa, Bordetella pertussis
MLINNKKLLHHILPILVLALLGMRTAQAVAPGIVIPPKALFTQQGGAYGRCPNGTRALTVAELRGNAELQTYLRQITPGWSIYGLYDGTYLGQAYGGIIKDAPPGAGFIYRETFCITTIYKTGQPAADHYYSKVTATRLLASTNSRLCAVFVRDGQSVIGACASPYEGRYRDMYDALRRLLYMIYMSGLAVRVHVSKEEQYYDYEDATFQTYALTGISLCNPAASIC

SEQ ID NO: 231 - protein, 76 aa, SARS coronavirus
MYSFVSEETGTLIVNSVLLFLAFVVFLLVTLAILTALRLCAYCCNIVNVSLVKPTVYVYSRVKNLNSSEGVPDLLV

SEQ ID NO: 232 - protein, 125 aa, Homo sapiens
QIKLMLENRAMVRRKGFGILIGVYGSSVIYMVQLPIFGVIDTPCWIIKAAPSCSEKDGNYACLLREDQGWYCKNAGSTVYYPNEKDCETRGDHVFCDTAAGINVAEQSRECNINISTTNYPCKVS

SEQ ID NO: 233 - protein, 221 aa, SARS coronavirus
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 234 - protein, 422 aa, SARS coronavirus
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 235 - protein, 1255 aa, Human Immunodeficiency Virus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDFGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 236 - protein, 28 aa, Human Immunodeficiency Virus
BTTMFFRMPQDLNTMLNTVGGHQAAMQM

SEQ ID NO: 237 - protein, 20 aa, Human Immunodeficiency Virus
PQDLNTMLNTVGGHQAAMQM

SEQ ID NO: 238 - protein, 20 aa, Human Immunodeficiency Virus
GPIAPGQMREPRGSDIAGTT

SEQ ID NO: 239 - protein, 20 aa, Human Immunodeficiency Virus
GPIAPGQMREPRGSDIAGTT

SEQ ID NO: 240 - protein, 20 aa, Human Immunodeficiency Virus
KGCWKCGKEGHQMKDCTERQ

SEQ ID NO: 241 - protein, 20 aa, Human Immunodeficiency Virus
KGCWKCGKEGHQMKDCTERQ

SEQ ID NO: 242 - protein, 20 aa, Human Immunodeficiency Virus
HQMKDCTERQANFLGKIWPS

SEQ ID NO: 243 - protein, 20 aa, Human Immunodeficiency Virus
HQMKDCTERQANFLGKIWPS

SEQ ID NO: 244 - protein, 20 aa, Human Immunodeficiency Virus
HQMKDCTERQANFLGKIWPS

SEQ ID NO: 245 - protein, 20 aa, Human Immunodeficiency Virus
ATLEEMMTACQGVGGPGHKA

SEQ ID NO: 246 - protein, 20 aa, Human Immunodeficiency Virus
ATLEEMMTACQGVGGPGHKA

SEQ ID NO: 247 - protein, 21 aa, Human Immunodeficiency Virus
CQGVGGPGHKARVLAEAMSQV

SEQ ID NO: 248 - protein, 21 aa, Human Immunodeficiency Virus
CQGVGGPGHKARVLAEAMSQV

SEQ ID NO: 249 - protein, 20 aa, Human Immunodeficiency Virus
MFSALSEGATPQDLNTMLNT

SEQ ID NO: 250 - protein, 20 aa, Human Immunodeficiency Virus
MFSALSEGATPQDLNTMLNT

SEQ ID NO: 251 - protein, 20 aa, Human Immunodeficiency Virus
SILDIRQGPKEPFRDYVDRF

SEQ ID NO: 252 - protein, 20 aa, Human Immunodeficiency Virus
SILDIRQGPKEPFRDYVDRF

SEQ ID NO: 253 - protein, 20 aa, Human Immunodeficiency Virus
EPFRDYVDRFYKTLRAEQAS

SEQ ID NO: 254 - protein, 20 aa, Human Immunodeficiency Virus
EPFRDYVDRFYKTLRAEQAS

SEQ ID NO: 255 - protein, 20 aa, Human Immunodeficiency Virus
LLVQNANPDCKTILKALGPA

SEQ ID NO: 256 - protein, 20 aa, Human Immunodeficiency Virus
LLVQNANPDCKTILKALGPA

SEQ ID NO: 257 - protein, 20 aa, Human Immunodeficiency Virus
KTILKALGPAATLEEMMTAC

SEQ ID NO: 258 - protein, 20 aa, Human Immunodeficiency Virus
KTILKALGPAATLEEMMTAC

SEQ ID NO: 259 - protein, 20 aa, Human Immunodeficiency Virus
KTILKALGPAATLEEMMTAC

SEQ ID NO: 260 - protein, 20 aa, Human Immunodeficiency Virus
MGARASVLSGGELDRWEKIR

SEQ ID NO: 261 - protein, 20 aa, Human Immunodeficiency Virus
MGARASVLSGGELDRWEKIR

SEQ ID NO: 262 - protein, 20 aa, Human Immunodeficiency Virus
GELDRWEKIRLRPGGKKKYK

SEQ ID NO: 263 - protein, 20 aa, Human Immunodeficiency Virus
GELDRWEKIRLRPGGKKKYK

SEQ ID NO: 264 - protein, 20 aa, Human Immunodeficiency Virus
LRPGGKKKYKLKHIVWASRE

SEQ ID NO: 265 - protein, 20 aa, Human Immunodeficiency Virus
LRPGGKKKYKLKHIVWASRE

SEQ ID NO: 266 - protein, 20 aa, Human Immunodeficiency Virus
LKHIVWASRELERFAVNPGL

SEQ ID NO: 267 - protein, 20 aa, Human Immunodeficiency Virus
LKHIVWASRELERFAVNPGL

SEQ ID NO: 268 - protein, 20 aa, Human Immunodeficiency Virus
LERFAVNPGLLETSEGCRQI

SEQ ID NO: 269 - protein, 20 aa, Human Immunodeficiency Virus
LERFAVNPGLLETSEGCRQI

SEQ ID NO: 270 - protein, 20 aa, Human Immunodeficiency Virus
LETSEGCRQILGQLQPSLQT

SEQ ID NO: 271 - protein, 20 aa, Human Immunodeficiency Virus
LETSEGCRQILGQLQPSLQT

SEQ ID NO: 272 - protein, 20 aa, Human Immunodeficiency Virus
LGQLQPSLQTGSEELRSLYN

SEQ ID NO: 273 - protein, 20 aa, Human Immunodeficiency Virus
LGQLQPSLQTGSEELRSLYN

SEQ ID NO: 274 - protein, 20 aa, Human Immunodeficiency Virus
GSEELRSLYNTVATLYCVHQ

SEQ ID NO: 275 - protein, 20 aa, Human Immunodeficiency Virus
GSEELRSLYNTVATLYCVHQ

SEQ ID NO: 276 - protein, 20 aa, Human Immunodeficiency Virus
TVATLYCVHQRIEIKDTKEA

SEQ ID NO: 277 - protein, 20 aa, Human Immunodeficiency Virus
TVATLYCVHQRIEIKDTKEA

SEQ ID NO: 278 - protein, 20 aa, Human Immunodeficiency Virus
RIEIKDTKEALDKIEEEQNK

SEQ ID NO: 279 - protein, 20 aa, Human Immunodeficiency Virus
RIEIKDTKEALDKIEEEQNK

SEQ ID NO: 280 - protein, 20 aa, Human Immunodeficiency Virus
LDKIEEEQNKSKKKAQQAAA

SEQ ID NO: 281 - protein, 20 aa, Human Immunodeficiency Virus
LDKIEEEQNKSKKKAQQAAA

SEQ ID NO: 282 - protein, 20 aa, Human Immunodeficiency Virus
SKKKAQQAAADTGHSNQVSQ

SEQ ID NO: 283 - protein, 20 aa, Human Immunodeficiency Virus
SKKKAQQAAADTGHSNQVSQ

SEQ ID NO: 284 - protein, 20 aa, Human Immunodeficiency Virus
DTGHSNQVSQNYPIVQNIQG

SEQ ID NO: 285 - protein, 20 aa, Human Immunodeficiency Virus
DTGHSNQVSQNYPIVQNIQG

SEQ ID NO: 286 - protein, 20 aa, Human Immunodeficiency Virus
NYPIVQNIQGQMVHQAISPR

SEQ ID NO: 287 - protein, 20 aa, Human Immunodeficiency Virus
NYPIVQNIQGQMVHQAISPR

SEQ ID NO: 288 - protein, 20 aa, Human Immunodeficiency Virus
QMVHQAISPRTLNAWVKVVE

SEQ ID NO: 289 - protein, 20 aa, Human Immunodeficiency Virus
QMVHQAISPRTLNAWVKVVE

SEQ ID NO: 290 - protein, 20 aa, Human Immunodeficiency Virus
TLNAWVKVVEEKAFSPEVIP

SEQ ID NO: 291 - protein, 20 aa, Human Immunodeficiency Virus
TLNAWVKVVEEKAFSPEVIP

SEQ ID NO: 292 - protein, 20 aa, Human Immunodeficiency Virus
EKAFSPEVIPMFSALSEGAT

SEQ ID NO: 293 - protein, 20 aa, Human Immunodeficiency Virus
EKAFSPEVIPMFSALSEGAT

SEQ ID NO: 294 - protein, 20 aa, Human Immunodeficiency Virus
VGGHQAAMQMLKETINEEAA

SEQ ID NO: 295 - protein, 20 aa, Human Immunodeficiency Virus
VGGHQAAMQMLKETINEEAA

SEQ ID NO: 296 - protein, 20 aa, Human Immunodeficiency Virus
LKETINEEAAEWDRVHPVHA

SEQ ID NO: 297 - protein, 20 aa, Human Immunodeficiency Virus
LKETINEEAAEWDRVHPVHA

SEQ ID NO: 298 - protein, 20 aa, Human Immunodeficiency Virus
EWDRVHPVHAGPIAPGQMRE

SEQ ID NO: 299 - protein, 20 aa, Human Immunodeficiency Virus
EWDRVHPVHAGPIAPGQMRE

SEQ ID NO: 300 - protein, 20 aa, Human Immunodeficiency Virus
PRGSDIAGTTSTLQEQIGWM

SEQ ID NO: 301 - protein, 20 aa, Human Immunodeficiency Virus
PRGSDIAGTTSTLQEQIGWM

SEQ ID NO: 302 - protein, 20 aa, Human Immunodeficiency Virus
STLQEQIGWMTNNPPIPVGE

SEQ ID NO: 303 - protein, 20 aa, Human Immunodeficiency Virus
STLQEQIGWMTNNPPIPVGE

SEQ ID NO: 304 - protein, 20 aa, Human Immunodeficiency Virus
TNNPPIPVGEIYKRWIILGL

SEQ ID NO: 305 - protein, 20 aa, Human Immunodeficiency Virus
TNNPPIPVGEIYKRWIILGL

SEQ ID NO: 306 - protein, 20 aa, Human Immunodeficiency Virus
IYKRWIILGLNKIVRMYSPT

SEQ ID NO: 307 - protein, 20 aa, Human Immunodeficiency Virus
IYKRWIILGLNKIVRMYSPT

SEQ ID NO: 308 - protein, 20 aa, Human Immunodeficiency Virus
NKIVRMYSPTSILDIRQGPK

SEQ ID NO: 309 - protein, 20 aa, Human Immunodeficiency Virus
NKIVRMYSPTSILDIRQGPK

SEQ ID NO: 310 - protein, 20 aa, Human Immunodeficiency Virus
YKTLRAEQASQEVKNWMTET

SEQ ID NO: 311 - protein, 20 aa, Human Immunodeficiency Virus
YKTLRAEQASQEVKNWMTET

SEQ ID NO: 312 - protein, 20 aa, Human Immunodeficiency Virus
QEVKNWMTETLLVQNANPDC

SEQ ID NO: 313 - protein, 20 aa, Human Immunodeficiency Virus
QEVKNWMTETLLVQNANPDC

SEQ ID NO: 314 - protein, 15 aa, Human Immunodeficiency Virus
RVLAEAMSQVTNSAT

SEQ ID NO: 315 - protein, 15 aa, Human Immunodeficiency Virus
RVLAEAMSQVTNSAT

SEQ ID NO: 316 - protein, 20 aa, Human Immunodeficiency Virus
TNSATIMMQRGNFRNQRKIV

SEQ ID NO: 317 - protein, 20 aa, Human Immunodeficiency Virus
TNSATIMMQRGNFRNQRKIV

SEQ ID NO: 318 - protein, 20 aa, Human Immunodeficiency Virus
GNFRNQRKIVKCFNCGKEGH

SEQ ID NO: 319 - protein, 20 aa, Human Immunodeficiency Virus
GNFRNQRKIVKCFNCGKEGH

SEQ ID NO: 320 - protein, 20 aa, Human Immunodeficiency Virus
KCFNCGKEGHTARNCRAPRK

SEQ ID NO: 321 - protein, 20 aa, Human Immunodeficiency Virus
KCFNCGKEGHTARNCRAPRK

SEQ ID NO: 322 - protein, 20 aa, Human Immunodeficiency Virus
TARNCRAPRKKGCWKCGKEG

SEQ ID NO: 323 - protein, 20 aa, Human Immunodeficiency Virus
TARNCRAPRKKGCWKCGKEG

SEQ ID NO: 324 - protein, 20 aa, Human Immunodeficiency Virus
ANFLGKIWPSYKGRPGNFLQ

SEQ ID NO: 325 - protein, 20 aa, Human Immunodeficiency Virus
ANFLGKIWPSYKGRPGNFLQ

SEQ ID NO: 326 - protein, 18 aa, Human Immunodeficiency Virus
YKGRPGNFLQSRPEPTAP

SEQ ID NO: 327 - protein, 18 aa, Human Immunodeficiency Virus
YKGRPGNFLQSRPEPTAP

SEQ ID NO: 328 - protein, 20 aa, Human Immunodeficiency Virus
SRPEPTAPPEESFRSGVETT

SEQ ID NO: 329 - protein, 20 aa, Human Immunodeficiency Virus
SRPEPTAPPEESFRSGVETT

SEQ ID NO: 330 - protein, 21 aa, Human Immunodeficiency Virus
PEESFRSGVETTTPPQKQEPI

SEQ ID NO: 331 - protein, 21 aa, Human Immunodeficiency Virus
PEESFRSGVETTTPPQKQEPI

SEQ ID NO: 332 - protein, 20 aa, Human Immunodeficiency Virus
TTPPQKQEPIDKELYPLTSL

SEQ ID NO: 333 - protein, 20 aa, Human Immunodeficiency Virus
TTPPQKQEPIDKELYPLTSL

SEQ ID NO: 334 - protein, 21 aa, Human Immunodeficiency Virus
DKELYPLTSLRSLFGNDPSSQ

SEQ ID NO: 335 - protein, 21 aa, Human Immunodeficiency Virus
DKELYPLTSLRSLFGNDPSSQ

SEQ ID NO: 336 - protein, 20 aa, Simian Immunodeficiency Virus
ATETLAGAWGDLWETLRRGG

SEQ ID NO: 337 - protein, 20 aa, Simian Immunodeficiency Virus
DLWETLRRGGRWILAIPRRI

SEQ ID NO: 338 - protein, 19 aa, Simian Immunodeficiency Virus
RWILAIPRRIRQGLELTLL

SEQ ID NO: 339 - protein, 500 aa, Human Immunodeficiency Virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 340 - protein, 879 aa, Simian Immunodeficiency Virus
MGCLGNQLLIAILLLSVYGTYCTLYVTVFYGVPAWRNATIPLFCATKNRDTWGTTQCLPDNGDYSELALNVTESFDAWNNTVTEQAIEDVWQLFETSIKPCVKLSPLCITMRCNKSETDRWGLTKSITTTASTTSTTASAKVDMVNETSSCIAQDNCTGLEQEQMISCKFNMTGLKRDKKKEYNETWYSADLVCEQGNSTGNESRCYMNHCNTSVIQESCDKHYWDAIRFRYCAPPGYALLRCNDTNYSGFMPKCSKVVVSSCTRMMETQTSTWFGFNGTRAENRTYIYWHGKDNRTIISLNKYYNLTIKCRRPGNKTVLPVTIMSGLVFHSQPINDRPKQAWCWFGGKWKDAIKEVKQTIVKHPRYTGTNDTARINLTAPGGGDPEVTFMWTNCRGEFLYCKMNWFLNWVEDRNTTNQKPKEQYKRNYVPCHIRQIINTWHKVGKNVYLPPREGDLTCNSTVTSLIANIDWIDGNQTNITMSAEVAELYRLELGDYKLVEITPIGLAPTNVKRYTTGGTSRNKRGVFVLGFLGFLATAGSAMGAASLTLTAQSRTLLAGIVQQQQQLLDVVKRQQELLRLTVWGTKNLQTRVTAIEKYLKDQAQLNAWGCAFRQVCHTTVPWPNTSLTPKWDNETWQEWERKVDFLEENITALPEEAQIQQEKNMYELQKLNSWDVFGNWFDLASWIKYIQYGVYIVVGVILLRIVIYIVQMLAKLRQGYRPVFSSPPSYFQQTHIQQDPALPTREGKEGDGGEGDGNSSWPWQIEYIHLLIRQLIRLLTWLFSNCRTLLSRVYQILQPILQRLSATLQRIREVLRTELTYLQYGWSYFHEAVQAAWRSATETLAGAWGDLWETLRRGGRWILAIPRRIRQGLELTLL